(19) United States
(12) Patent Application Publication
(10) Pub. No.: US 2017/0156723 A1
(43) Pub. Date: Jun. 8, 2017

(54) ANCHOR DELIVERY SYSTEM

(71) Applicant: Neosurgical Limited, Dublin (IE)

(72) Inventors: Ronan Keating, Parkmore (IE); Gerard Rabbitte, Parkmore (IE); Diarmuid Conroy, Parkmore (IE); Stephen Williams, Blackrock (IE)

(21) Appl. No.: 15/320,268

(22) PCT Filed: Jun. 19, 2015
(86) PCT No.: PCT/EP2015/063886

§ 371 Date: Dec. 19, 2016

(30) Foreign Application Priority Data
Jun. 19, 2014 (GB) 1410977.1

(51) Int. Cl. A61B 17/064; A61B 17/04

(52) U.S. Cl. CPC A61B 17/064; A61B 17/0401; A61B 2017/0409; A61B 2017/0417; A61B 2017/0419; A61B 2017/0464; A61B 2017/0647; A61F 2/0063

(57) ABSTRACT

An anchor delivery system is described. The system includes an anchor and a cooperating driver, the driver facilitating storage and delivery of the anchor/anchors into the abdominal cavity and subsequently placement of the anchor in the inner abdominal wall.

### FIELD OF THE INVENTION

The present invention relates to an anchor delivery system.

### BACKGROUND

Mesh placement is a common treatment in hernia repair and is increasingly completed laparoscopically. The procedure involves the surgeon first removing tissue from the defect in the abdominal wall to expose the opening. A repair mesh is then sized and inserted into the abdominal space. The mesh is then unfurled and positioned over the defect using graspers. Once the mesh is in position it is typically tacked in place using multiple tacks with a tacking device. Many known tacker devices are prone to jamming and some devices rely on the application of counter pressure on the external surface of the abdomen. A significant portion of the cost associated with tack delivery systems can be the absorbable material used to form the tack, these devices do not come a flexible number of tacks and often results in too many tacks being deployed or a fraction of the supplied tack being deployed. There are therefore a number of problems with current methods of mesh tacking that need to be addressed.

### SUMMARY

Accordingly, a first embodiment of the application provides a device as detailed in the independent claims. Advantageous embodiments are provided in the dependent claims.

A device per the present teaching may advantageously be used with a tack or anchor that are based on a two part construction, each of the two parts being coupled to one another using a flexible member. On deployment, one of the parts is placed in the abdominal wall, and the second part forms an abutment against the inner surface of the mesh so as to secure the mesh to the abdominal wall. This method of affixation may produce a lower tension fixation, and may require less tacks as the tacks are more secure, and hence is advantageous over prior art implementations.

### BRIEF DESCRIPTION OF THE DRAWINGS

The present invention will now be described with reference to the accompanying drawings in which:

FIGS. 1A to 1C show examples of a tacking device provided in accordance with the present teaching in a top view (FIG. 1A), a side view (FIG. 1B) and in cross section (FIG. 1C)

FIGS. 2A and 2B show detail of an actuation mechanism that may be used with a tacking device per FIG. 1C.

FIGS. 3A, 3B and 3C show another example of a tacking device in accordance with the present teaching.

FIGS. 4A, 4B and 4C provide examples of a tacking device in accordance with the present teaching.

FIGS. 5A, 5B and 5C provide examples of example of a tacking device in accordance with the present teaching.

FIGS. 6A, 6B and 6C provide examples of example of a tacking device in accordance with the present teaching.

FIGS. 7A, 7B, 7C and 7D provide examples of a tack that may be used in accordance with the present teaching.

FIGS. 8A, 8B, 8C and 8D provide examples of a tack that may be used in accordance with the present teaching.

FIGS. 9A and 9B provide examples of distal portion of a device that may be used in accordance with the present teaching.

FIG. 9C shows three examples of an end view of the distal portion shown in FIGS. 9A and 9B including inserts that are used to effect delivery of an anchor through the device.

FIGS. 10A, 10B and 10C and 10D provide examples of distal portion of a device that may be used in accordance with the present teaching.

FIGS. 11 and 12 shows another example of a tacking device in accordance with the present teaching.

FIGS. 13A and 13B shows further detail of the device from FIGS. 11 and 12 in accordance with the present teaching.

FIG. 14 provides an example of a tack that may be used in accordance with the present teaching.

FIGS. 15A to 15E shows another example of a tacking device in accordance with the present teaching both in perspective and sectional views.

FIGS. 16A to 16B and 16C provide another example of a tacking device in accordance with the present teaching.

FIGS. 17A, 17B and 17C and 17D provides further examples of a tack that may be used in accordance with the present teaching.

FIGS. 18A, 18B, 18C, 18D and 18E provide further examples of a tack that may be used in accordance with the present teaching.

FIGS. 19A and 19B show another example of a tacking device in accordance with the present teaching.

FIGS. 19C and 19D show another example of an anchor or tack (FIG. 19C) and a cooperating tacking device (FIG. 19D) in accordance with the present teaching.

FIGS. 20A and 20B show additional views of the device from FIGS. 19A and 19B in pre and post deployment states.

FIGS. 21A and 21B show additional views of the device from FIGS. 19C and 19D in pre and post deployment states.

FIGS. 22A to 22F show the devices of 19A to 19D being used with a reloadable cartridge.

FIGS. 23A and 23B illustrated further examples of a tack and the distal portion of a device that such a tack would be used with.

FIG. 24 illustrates another example of a tack and how such a tack might be used to engage with the abdominal wall.

FIG. 25 shows a device similar to that described in FIG. 2, but modified to be supplied as a two component part comprising a handle portion and a cartridge portion.

FIG. 26A shows an uncoupled example of an interlocking mechanism that could be used with the device of FIG. 25, while the same mechanism is shown coupled in FIG. 26B.

FIG. 27A shows an uncoupled example of another interlocking mechanism that could be used with the device of FIG. 25, while the same mechanism is shown coupled in FIG. 27B.

FIGS. 28A and 28B show the hidden detail for the mechanism of FIG. 27.

FIG. 29 shows in partly-sectional view a further example of a tacking device in accordance with the present teaching.

FIG. 30 shows an exploded view of the tacking device of FIG. 29.

FIG. 31 shows a plurality of examples of tacks that may be used within the context of a device per FIG. 29.

FIG. 32 shows further views of the device of FIG. 29 with exploded views of both a cartridge and the handle distal portion of the shaft.

FIG. 33A shows the device of FIG. 29 in a ready to deploy tack configuration.

FIG. 33B is a perspective of a cartridge that may be usefully employed with the device of FIG. 33A.

FIG. 33C is a section view of the cartridge of FIG. 33B.

FIGS. 34A to 34C show examples of another tacking device in accordance with the present teaching wherein a two part tack or anchor is partially disposed within the shaft of the device during delivery.

FIGS. 35A, 35B and 36 show another example of a tacking device in accordance with the present teaching that uses a replaceable tack cartridge that operably rotates to provide sequential tacks for delivery through the shaft of the tacking device.

FIG. 37 shows additional variants to an anchor that may be usefully employed within the context of the present teaching.

FIG. 38 is an example of a delivery device that may be usefully employed with the anchors of FIG. 37.

FIG. 39 is a further example of a delivery device, similar to that of FIG. 33 but modified to accommodate larger dimensioned second part anchors.

### DETAILED DESCRIPTION OF THE DRAWINGS

The teaching of the present invention will now be described with reference to exemplary embodiments thereof which are provided to assist with an understanding of the present teaching and are not to be construed as limiting in any way. It will be appreciated that modifications can be made to the exemplary arrangements which follow without departing from the spirit or scope which is only to be limited insofar as is deemed necessary in the light of the appended claims.

Within the context of the present teaching an anchor delivery system advantageously allows for the delivery of an anchor within an abdominal cavity of the patient. Within the context of the present teaching the terms “anchor” and “tack” will be used interchangeably. Where a plurality of anchors are used for fixation purposes, for example the fixation of a mesh within the abdominal cavity they will typically be referred to as tacks. Where one or more anchors are used to secure a surgical device within the abdominal cavity or effect closure of a wound they will typically be referred to as anchors.

It will be appreciated that the following discussion regarding the specifics of the abdominal cavity and abdominal wall should not be construed as limiting in that a system provided in accordance with the present teaching may be used with other types of tissue including but not limited to organs, bones or the like. The use of an anchor delivery system per the present teaching can be used for one or more of anchoring laparoscopic surgical equipment, assisting in the moving of internal organs to allow a surgeon access to a surgical site, or closure of a wound post completion of a surgical procedure. In such a latter configuration, where the suture is coupled to an anchor, as the suture is passed through the abdominal wall and is held within the wall by the anchors that will remain deployed within the abdominal cavity, a subsequent tightening of the sutures will cause the sides of the incision, or defect in the abdominal cavity to be brought together to close the wound. The adoption of such a technique will advantageously require the use of bioabsorbable anchors, as the anchors will remain within the abdominal cavity during the healing process prior to their ultimate disintegration.

In accordance with an aspect of the present teaching an anchor is provided in two parts that are coupled to one another but which are moveable relative to one another. This may be achieved by providing a first part coupled to a second part via a tether that may be a flexible tether. The tether or interconnect between the two parts may be formed from the same or different material to each of the first part and the second part. The anchor may then be delivered to the surgical site through co-operation of the anchor with a delivery tool. The delivery tool engages with the anchor and is then used to deliver the anchor through to the abdominal cavity. In certain configurations the anchor may be coupled to suture and such configurations are particularly advantageously used in the context of wound closure or retraction. Without use of suture such anchors or tacks are advantageously used as fixation devices.

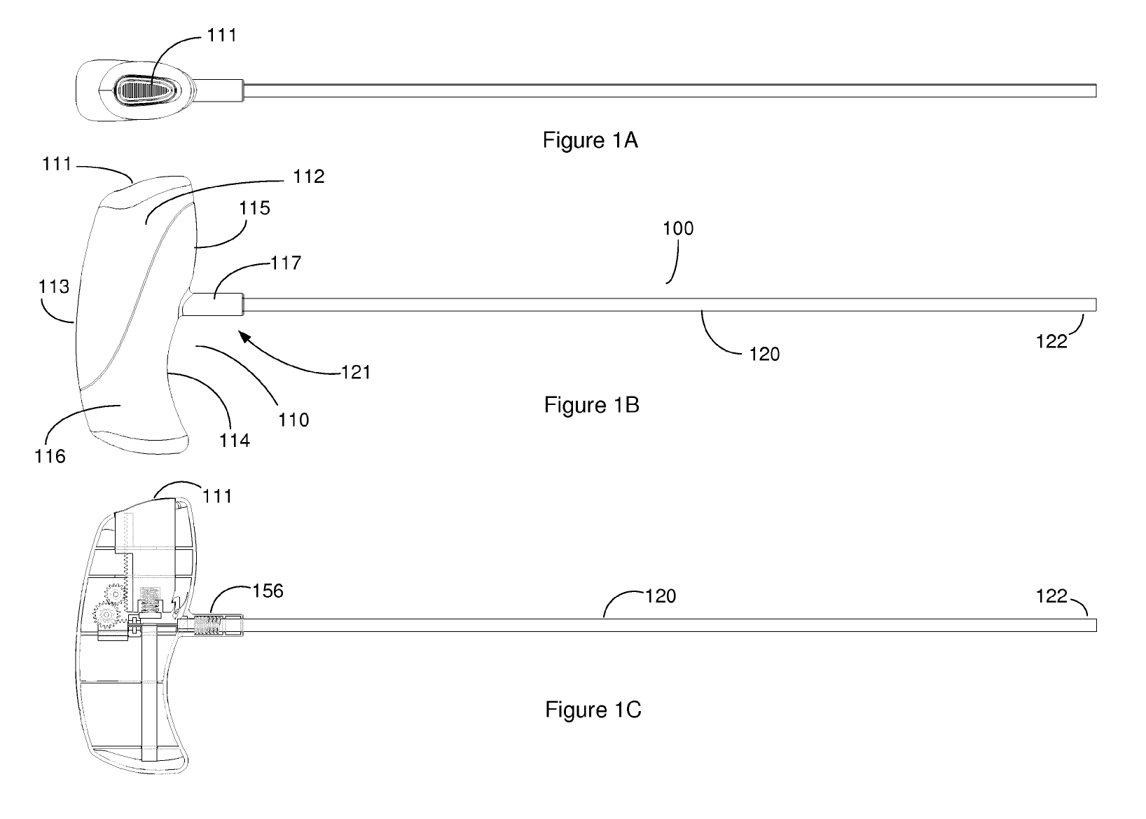

FIG. 1 illustrates device 100 which comprises a proximal handle 110 and a shaft 120. The handle comprises a surface 113 which will abut with the palm of a user's hand in use and surfaces 114 and 115 against which the fingers will engage. The handle features a trigger 111, which is normally in a non-active state. The shaft 120 has a handle distal end of the shaft 122 and a handle proximal end of the shaft 117. As is seen in the sectional view of FIG. 1C, the shaft 120 is coupled to the handle via a spring 156 so as to be moveable relative to the handle 110. In this way when the handle distal end of the shaft 122 or shaft tip engages with the target inner abdominal wall surface, it retracts upon pressure being applied towards the handle, this retraction being provided by compression of the spring 156.

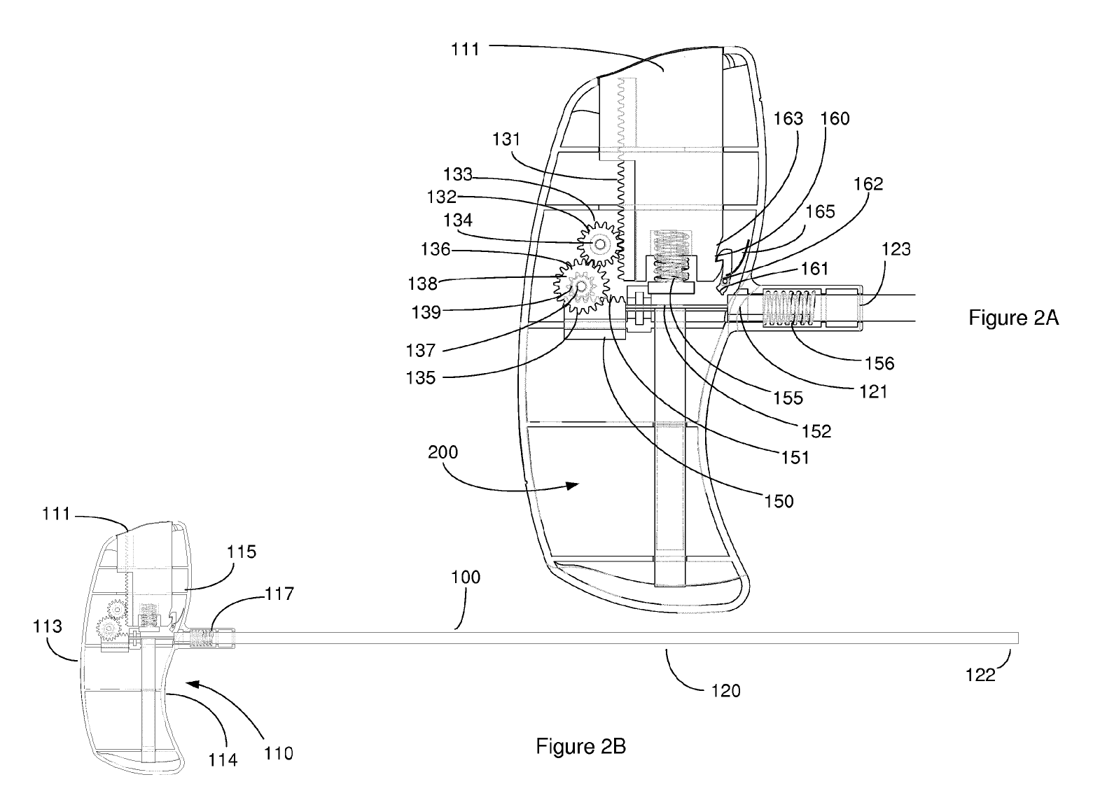

Turning now to FIG. 2A, the pressure at the tip causes the shaft, which is normally sprung in the forward position by spring 156, to move towards the proximal end. Once sufficient pressure is applied to the outer shaft a mechanism in the handle trips and the trigger 111 is exposed. This occurs when the proximal end of the shaft 121 engages with a bottom edge 161 of the trigger catch. The trigger button 111 is then released and a second spring 155 causes the trigger button to move up. This trigger is then available to be depressed when the user wishes to deploy the anchor, and prevents premature deployment of the anchor. The handle will ideally be manufactured from a rigid mouldable polymer such as a thermoplastic like Acrylonitrile butadiene styrene (ABS) for example, and may feature an area 112 which is moulded in a softer material, to provide grip.

The handle provides an interior hollow volume 200 dimensioned to accommodate additional components to control the trigger 111 and the pusher component 152 When the shaft 120 of FIG. 1 engages with a surface at the distal tip 122 and pressure is applied, the spring 156 of FIG. 2A is compressed and the handle moves forward relative to the shaft. The proximal end of the shaft within the handle 121 engages with the bottom edge 161 of the trigger catch. The trigger catch features a protrusion 160 which engages with a notch on the trigger 163, and is pivotable on the pivot 162. Movement of proximal end of the shaft into engagement with the bottom edge of the catch, causes the catch to disengage from the trigger. The trigger is then moved up by the spring 155. Once the shaft 120 moves forward, a flat spring 165 returns the trigger catch, and it can reengage the trigger, when the trigger is depressed, such that the notch on the trigger 163 is aligned with the protrusion on the trigger catch.

When the trigger is depressed a rack 131 engages with the teeth 133 of the gear wheel 132, and drives the wheel in a clockwise direction. The gear wheel 132 is pivoted about the pivot 134 and engages with gear wheel 136, which is pivoted about the pivot 137. This gear wheel is a stepped gear and is driven in a counter-clockwise direction and the teeth of the smaller diameter portion 138 engage with a rack 151, which is in turn connected to a pusher 152. The pusher engages at the distal end of the device with the proximal portion of an anchor 312.

Depression of the trigger causes counter-clockwise motion of the gear wheel 139, which causes the rack 151 to move to the right when looking at the image, and is equivalent to forward motion of the pusher. When the trigger catch is released and the trigger is deployed, the rack 131 engages with gear wheel 132. Movement of the rack 131 provides corresponding movement of the gear wheel in a counter clockwise direction. Gear wheel 132 also engages with gear wheel 136 and the counter clockwise rotation of 132 causes gear wheel 136 to move in a clockwise direction. As gear wheel 136 through the stepped gear 139 is also in contact with the pusher through the rack 151, motion of the gear wheel 136 causes the rack to move to the left as viewed. This causes the pusher to return to the start position, ready for deployment of the next anchor.

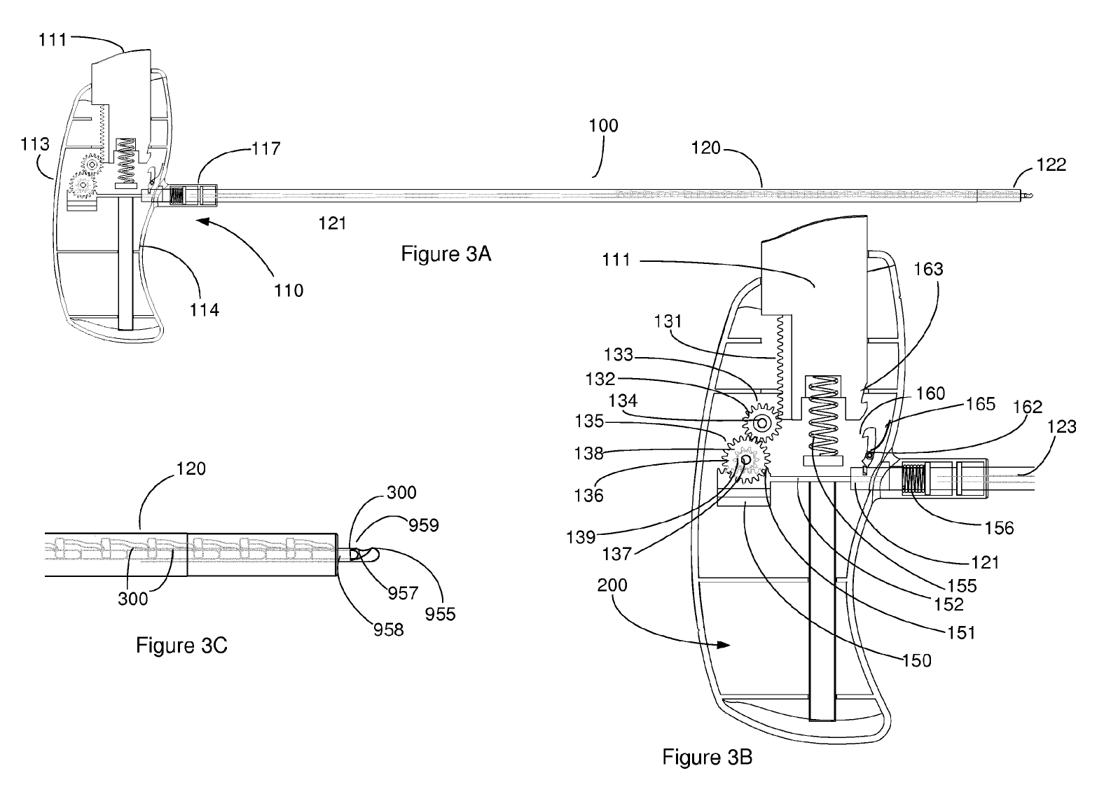

Turning now to FIGS. 3A, 3B and 3C, which show the device with the trigger deployed due to pressure applied at the distal tip 122 by the abdominal wall for example. Here the spring 155 is shown extended as it is forcing the trigger up. The needle tip 955 of the device is exposed from the distal tip of the device 122 and is driven into tissue as pressure is applied to the handle, with the shaft 120 moving back to reveal the tip. As the outer shaft moves back it compresses the spring 156. When pressure is released, by not pressing the device against the abdominal wall, the spring 156 will return to its uncompressed state and return the outer shaft over the tip to cover the needle tip. The needle tip 955 is provided at the distal end of a hollow needle shaft 958, which houses the anchor to be deployed. The shaft has a side opening 959 through which the anchor exits. The inner surface may feature a ramp to facilitate smooth movement of the anchor out of the side opening 959. The tip 955 is illustrated as an atraumatic blunt nosed tip, but could also feature a lancet, bevel or trocar tip geometry, and is not intended to be limiting.

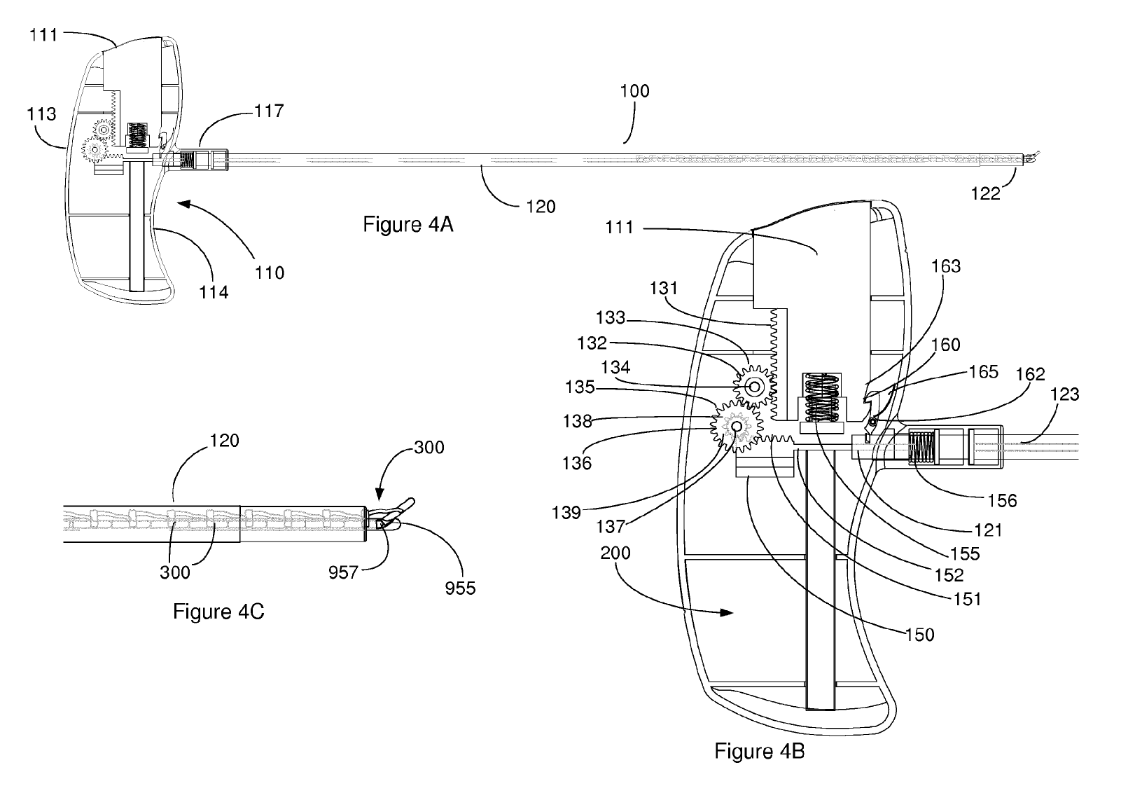

Continuing to FIG. 4 (FIGS. 4A, 4B, and 4C) which illustrates the effect of a depression of the trigger, here the spring 155 is compressed as the trigger has been depressed. The spring is prevented from returning to its uncompressed state by the trigger catch. The downwards movement of the trigger forces the pusher 152 forward as described in detail earlier. This causes the pusher to push the two part anchor 300 out of the needle shaft. The shaft features a ramp 957 which is designed to optimise the trajectory of the anchor into the tissue. Once the trigger is fully depressed, this is an indication to the user that the anchor has been deployed. Pressure on the handle is released, allowing the spring 156 to extend and the shaft 120 to cover the needle tip 955. In use when the shaft 120 is engaged with the abdominal wall the relative movement of the needle tip will translate to the needle tip withdrawing from the abdominal wall and into the tip 122 of the shaft 120.

For illustrative purposes a plurality of anchors are shown in FIG. 4C which are packed end to end in the needle shaft 958. For the embodiment illustrated in FIG. 1, where the pusher is advanced a certain distance before being retracted, due to the interaction of gears with racks it would be necessary to supply the anchor in a shaft which is substantially parallel to the needle shaft 958. In this scenario the anchors could be advanced by a spring. A side opening could be provided in the spring shaft, which would be opened, when the pusher move back, or to its home position. The anchors being sprung forward, would advance, such that a single anchor would be advanced into the needle shaft, ahead of the pusher. The pusher would then advance to deploy that anchor and prevent the next anchor entering the needle shaft until it is retracted. In this way sequential delivery of individual anchors may be provided.

An alternative solution would be a design where the pusher is continuously advanced, in the manner described under depression of the trigger, but that when the trigger is released the gear is disengaged, allowing for forward only movement of the pusher. To accommodate such a design, the pusher would need to extend in length, and protrude proximally from the surface 113 of the handle. This length extension could further be accommodated within the handle by utilizing a geared, spooled extrusion or by simply lengthening the handle.

A device per the present teaching may be provided with anchors having that are based on the principle of opposing T-bars which are coupled to one another using a flexible member or other interconnect to form a H-type structure. Each of the opposing T-bars or first and second parts of the anchor, can be used to secure a mesh within the abdominal cavity. The mesh is retained by having the first and second parts provided on opposing sides of the mesh.

These anchors, or tacks, are configured such that on deployment, one of the T-bars is placed in the abdominal wall, and by using a T-bar configuration once placed, the tack is securely retained within the abdominal wall. The opposing T-bar of the tack forms an abutment against the inner surface of the mesh so as to secure the mesh to the abdominal wall. Such T-bars could be constructed from two individual anchors such as described in our pending international application PCT/EP2012/072936 which are coupled together via a flexible member or tether. Each of the individual anchors when viewed absent to the other but when coupled to the flexible member adopts a T configuration. By attaching an anchor to each end of the flexible member, the configuration adopts an H configuration.

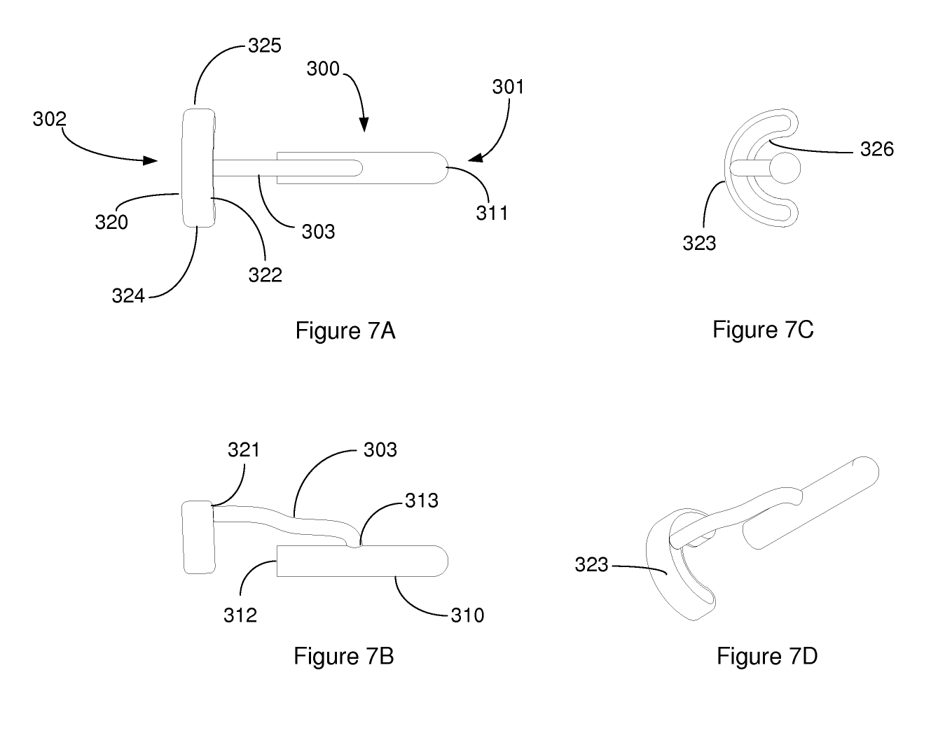

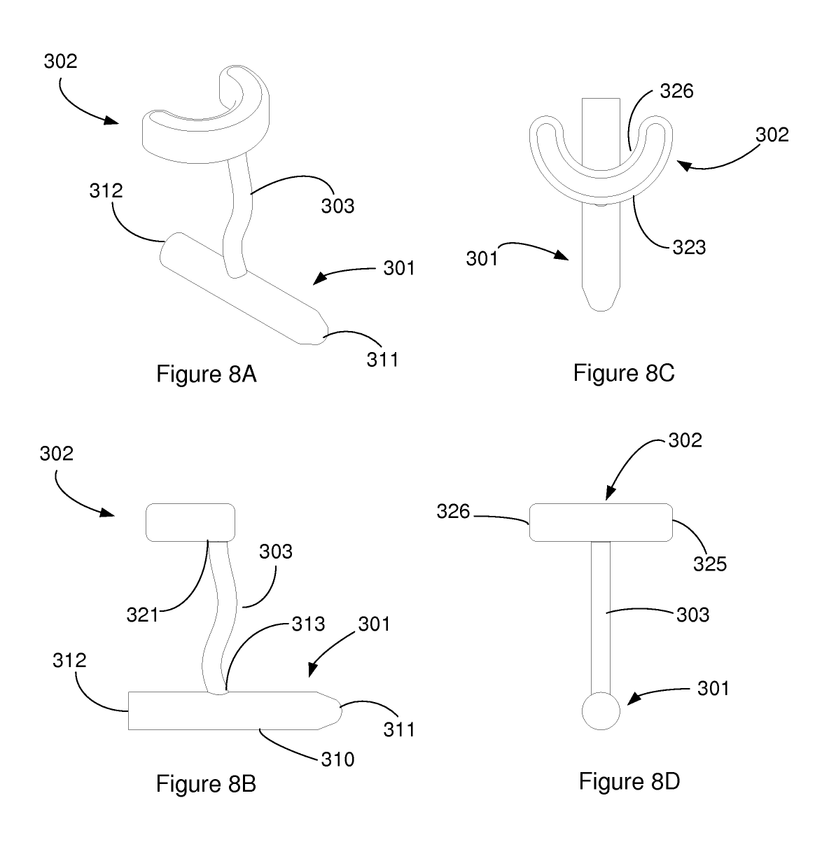

In a variant to this arrangement where the anchor is formed from first and second parts that have a similar geometric form, the present teaching provides anchors such as those described in FIG. 7 or FIG. 8. In this schematic the anchors are provided in a two part construct with first and second parts having a different geometrical form. A first part 300 has a tubular form and will operably be presented into the abdominal wall. To facilitate this presentation into the abdominal wall, this first part forms a distal end portion 301 of the anchor that terminates in a radiused or chamfered leading surface 311 which will be the first surface of the anchor to exit the driving tool. The leading edge 311 is profiled to ease insertion into tissue. While the leading edge is illustrated here as having a ball nose design it in not intended to be limiting and that the leading edge could be sharp with for example either a conical or bevelled end cut or configured to fit within the proximal lumen to facilitate engagement in a scenario where the anchors are loaded sequentially. The distal portion also comprises a non-leading edge 312. This portion of the anchor engages with the distal tip of the pusher 152 to advance the anchor from the device through tissue.

A second part of the anchor constitutes a proximal portion 302 of the anchor and is provided having a different geometrical form to that of the distal portion 301. The distal portion of the anchor is connected to the proximal portion 302 by an interconnect member 303 which may be flexible and which attaches to the distal portion approximately midway between the leading edge 311 and the non-leading edge 312 of the proximal portion 301, and midway between the edges 324 and 325 of the distal portion 302.

The proximal portion 302 has an inner curved surface 326 which is adopted to interact with an outer diameter surface of a delivery rod for example, while the outer curved surface 323 is intended to interact with an inner lumen of a delivery shaft, allowing the distal portion to be positioned in the annular space between a pusher rod and outer shaft. An advantage of such a design is that when the anchor is deployed, the surface area of the mesh engaging portion is increased, which means the load is disturbed over a larger area, which should reduce pain.

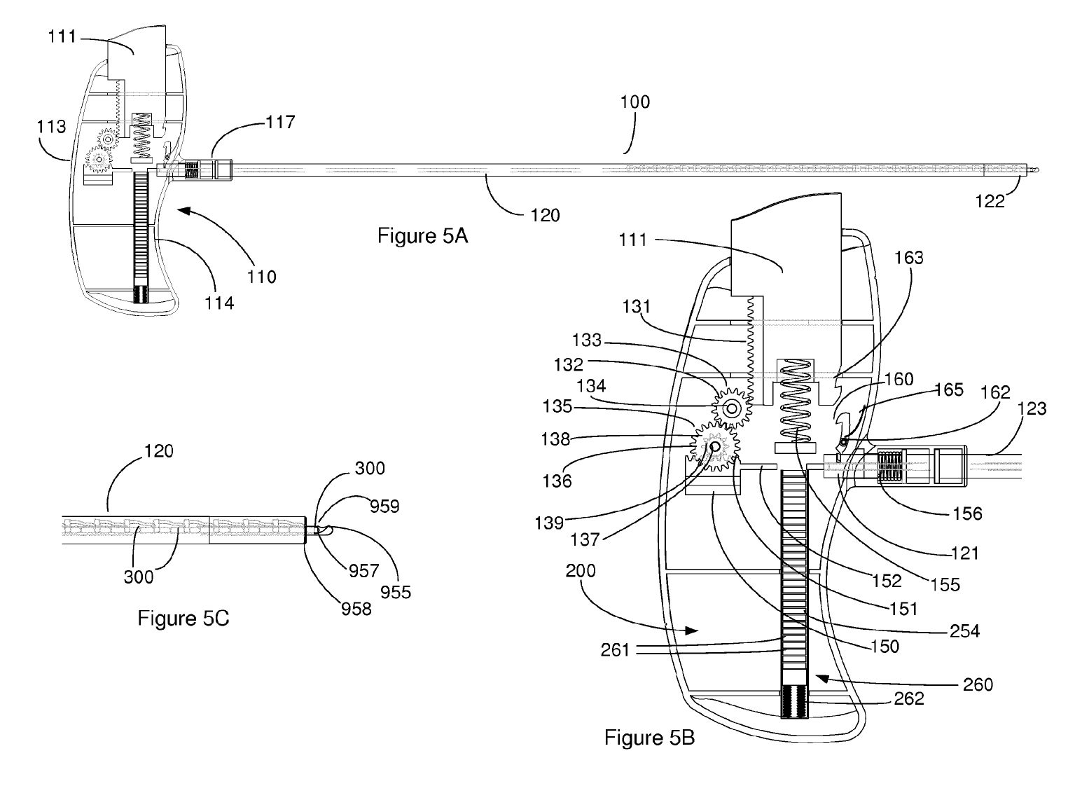

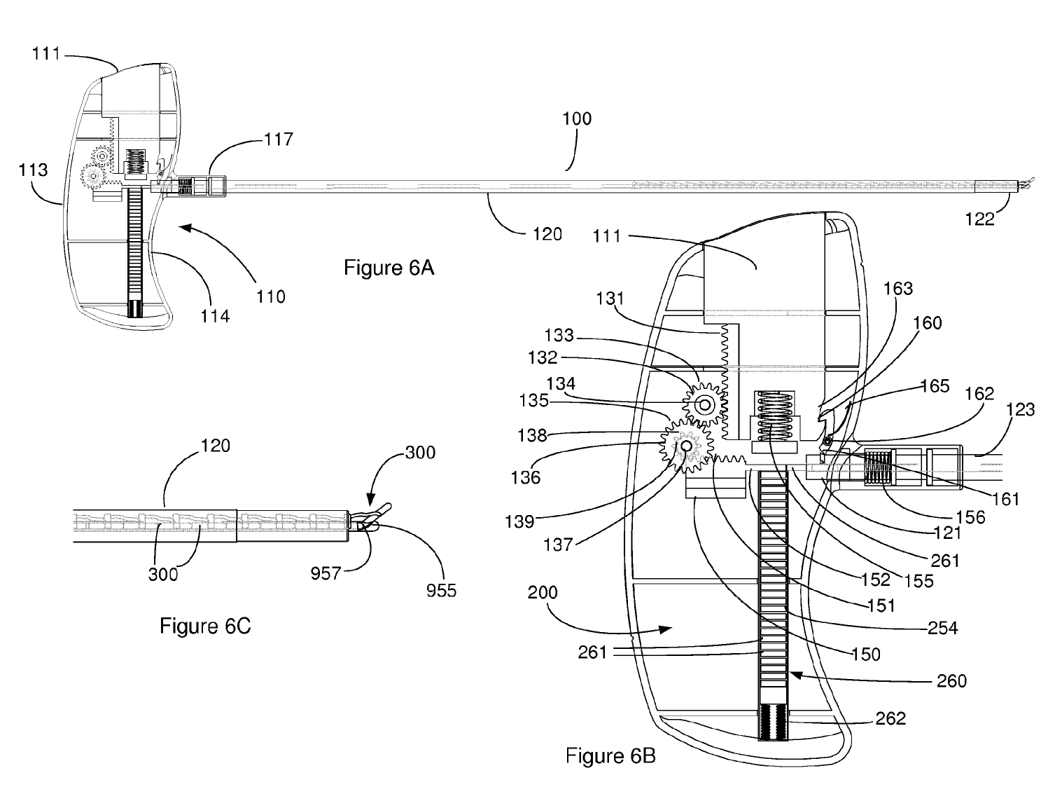

In a further embodiment of the device of FIG. 1 shown in FIGS. 5 (FIGS. 5A-5C) and FIG. 6 (FIGS. 6A-6C), the handle portion being numbered in a similar fashion to FIG. 1 for common components, a magazine 260 is filled with short lengths of plastic bead 261 which replicate the effect of, and could be substituted by, a ratchet mechanism. These lengths correspond to the stroke length required to deploy a single anchor. When the shaft 120 of FIG. 5 engages with a surface at the distal tip 122 and pressure is applied, the spring 156 is compressed and the handle moves forward relative to the shaft. The proximal end of the shaft within the handle 121 engages with the bottom edge 161 of the trigger catch. The trigger catch features a protrusion 160 which engages with a notch on the trigger 163, and is pivotable on the pivot 162. Movement of proximal end of the shaft into engagement with the bottom edge of the catch, causes it to disengage from the trigger. The trigger is the moved up by the spring 155 as illustrated in FIG. 5A. Once the shaft 120 moves forward, a spring 165 returns the trigger catch, and it can reengage the trigger, when the trigger is depressed.

When the trigger is depressed a rack 131 engages with the teeth 133 of the gear wheel, and drive the wheel in a clockwise direction. The gear wheel 132 is pivoted about the pivot 134 and engages with gear wheel 136, which is pivoted about the pivot 137. This gear wheel 136 features a step down gear and when driven in a counter-clockwise direction engages with a rack 151, which is in turn connected to a pusher. The counter-clockwise motion of the gear wheel 136 causes the rack to move to the right when looking at the image, and is equivalent to forward motion of the pusher. This pusher advances a length of plastic bead 261 which is housed in a magazine 254, and the forward motion of the pusher ejects a single anchor from the needle tip 955 of the device

The next time the tip of the shaft 122 is engaged with the abdominal wall trigger catch is released, and the spring 155 elongates to push up the trigger. This action causes the gear wheel 132 to engage with a rack 131 and as the trigger moves up the gear wheel is driven in a counter clockwise direction. Gear wheel 132 also engages with gear wheel 136 and the counter clockwise rotation of 132 causes gear wheel 136 to move in a clockwise direction. As smaller gear portion 139 of the gear wheel 136 is also in contact with the pusher through the rack 151, motion of the gear wheel causes the rack to move to the left as viewed and this causes the pusher to return to the start position. As the pusher retracts the next plastic bead 261 is pushed into the void by the compression spring 262, and is aligned, ready to be advanced by the pusher the next time the trigger 111 is activated.

In this arrangement the distal anchor portions 301 (as shown in FIG. 7A) of each anchor is in contact with the proximal portion 312 the next anchor and so on, with the proximal end of the proximal anchor being engaged by a pusher rod, such that forward motion is transmitted through the chain of anchors from the proximal most to the distal most. The distal anchor portion is held substantially parallel to the needle shaft lumen.

To use the devices of FIG. 2 or 5 the user:
a. Passes the device through a trocar or port until the distal tip of the device is in contact with the inner abdominal wall at the target location;
b. On application of sufficient pressure, the outer shaft retracts to expose the needle and the trigger on the handle is exposed and becomes active;
c. The user deploys an anchor at the desired location by depressing the trigger;
d. The device is then angled to the next desired location and the steps a to c are repeated to deploy the next anchor. This process is repeated until the user is satisfied with that sufficient affixation has been achieved, or the device is emptied of anchors;
e. The device is removed from the trocar or port;

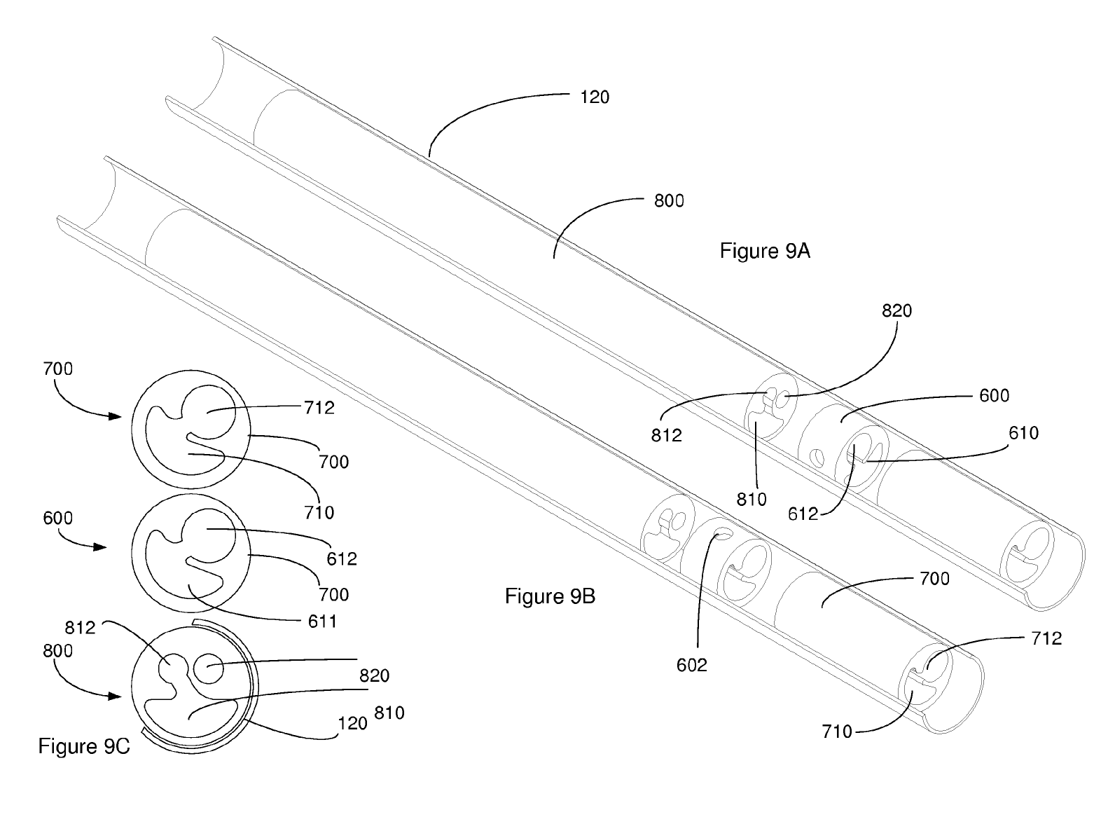

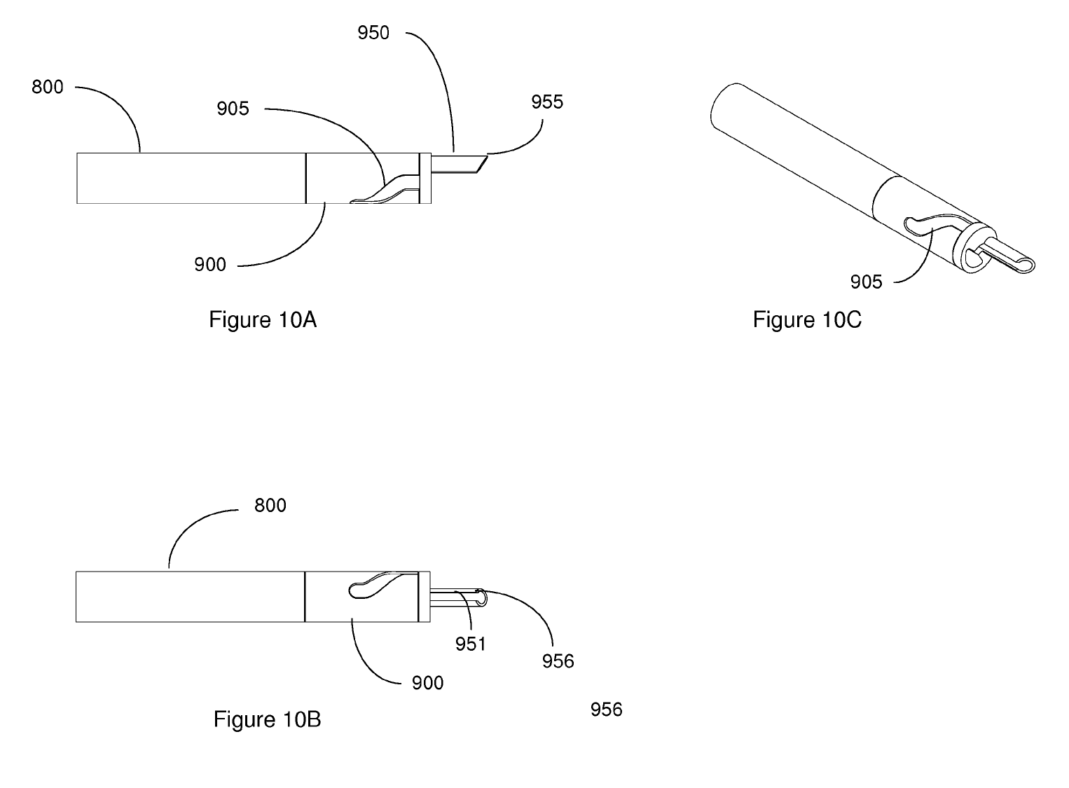

A further embodiment to the devices of FIGS. 2 and 5 is illustrated in FIGS. 9 and 10, where the proximal portion remains the same, but the distal portion is configured to facilitate moving an anchor from a storage channel to a deployment channel.

Turning first to FIG. 9A, the anchors—having a form similar to that described above with reference to FIG. 7 or 8—are loaded in the distal end of the shaft of the device 800 as opposed to through the handle portion. The proximal anchor portion 302 is loaded in the channel 810, while the distal portion of the anchor 301 is loaded into the channel 812. A pusher rod is disposed in channel 820 and is connected to the proximal end rack 151 (not shown in FIG. 9). In certain configurations multiple anchors can be placed in the storage channel 810/812 and the anchors are pushed forward by a spring (not shown). Of the multiple anchors in the storage channel, a single anchor is pushed into the portion 600, with the proximal anchor portion loaded into the channel 610 and the distal portion of the anchor loaded into 612. The distal portion of the anchor is prevented from moving forward by the protrusion 712 of the deployment channel portion 700. In this way the two part construct of the anchor is delivered through the driving tool in two distinct channels.

The delivery portion 600 serves to move the distal anchor portion in line with the pusher channel by rotation. As shown in a comparison of FIGS. 9A and 9B, the delivery portion 600 is moveable within the shaft 120 so as to bring the apertures 712, 612 and 812 into alignment with one another to allow delivery of an anchor through the distal end of the shaft. This movement is effected through use of a pin (not shown) which is placed in the hole 602 and which facilitates a movement of the delivery portion 600.

Turning now to FIG. 10, this pin engages with a curved slot 905 in the outer shaft to effect a rotation of the delivery portion 600, such that the channel 612 moves from being aligned with channel 812 to channel 820 and is now said to be in the delivery position. The portion 700 is aligned and fixed such that its channel 712 is aligned with 820, and channel 612 of delivery portion 600 when delivery portion 600 is in the delivery position. When the pusher advances the anchor moves first from channels 612 to 712 and out through the tip of the device 956 as the pusher engages with the proximal face 312 of the distal portion of the anchor. The trailing portion of the anchor 302 is pulled first from channel 610 through channel 710 and out through the tip of the device. The needle tip features a slot 951 which allows passage of the flexible member, which connects the proximal and distal portions of the anchor. In this way, while the first and second parts of the anchor are delivered separately in their own independent channels, prior to exiting the device they are biased into the same exit channel from which they will exit the device.

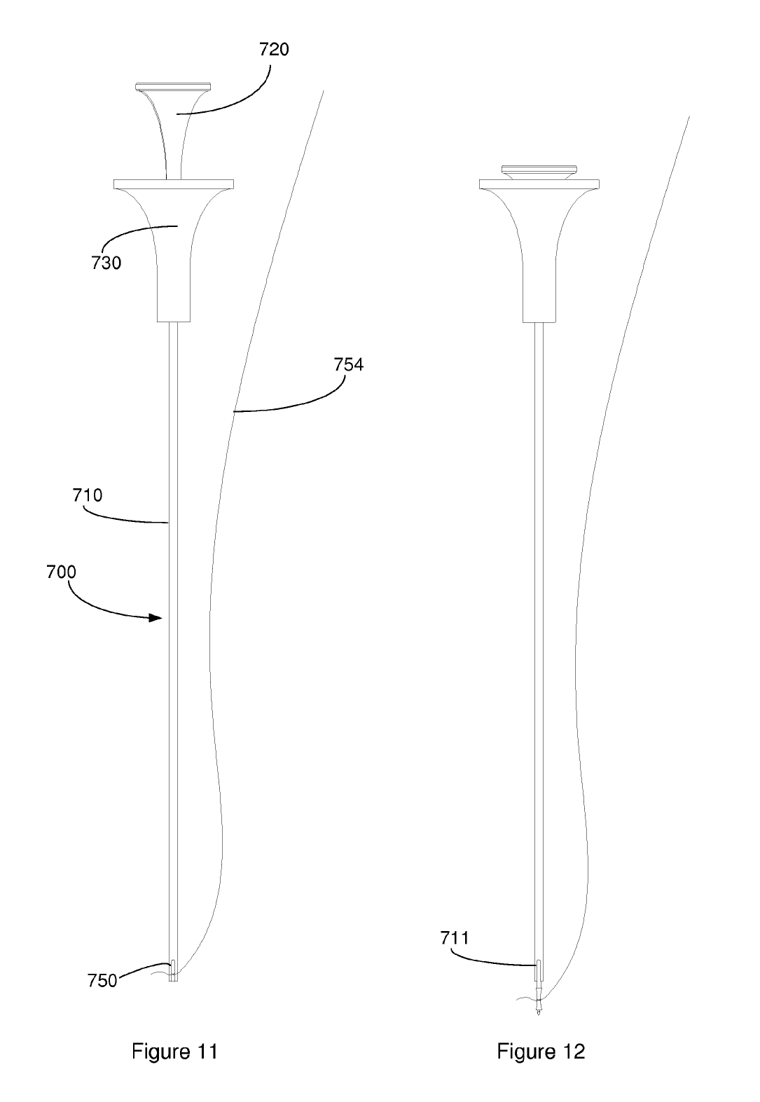

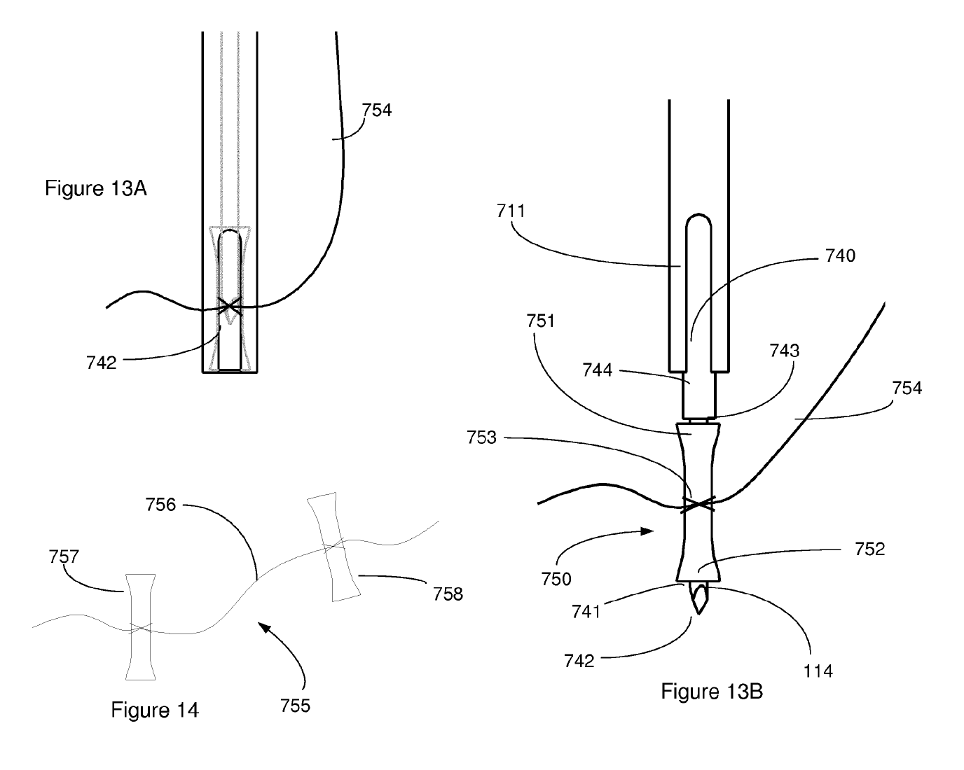

In another embodiment of the device illustrated as FIGS. 11 to 13 a single anchor 750 is loaded into the device. The anchor 750 is coupled to suture. The device comprises an outer shaft 710 which has a slot 711 at the distal end and is affixed to a handle portion 730. A wire 740 (FIG. 13B) runs through the lumen of this shaft and comprises a larger diameter portion 744 and a smaller diameter portion 71. The transition between these two diameters creates a step 743, which engages with the proximal end 751 of the anchor to engage the anchor and effect movement of the anchor concurrent with movement of the wire. The wire is affixed to the proximal handle portion 720 (FIG. 11). In a non-active state the wire tip 742 is hidden in the tip of the device inside the lumen of the anchor (FIG. 13A). When the handle portion 720 is compressed towards the handle portion 730, the wire is pushed forward through the anchor such that the needle tip is the leading cutting edge. The anchor is also advanced, as the step 743 engages with the anchor and forces it into the tissue though a pilot hole created by the wire tip 742. When the handle is released the wire retracts to its start position, as the handle is maintained in an open position by a compression spring inside the handle. The device can then be removed and be reloaded with another anchor.

This type of device could also be used with an anchor 755, as shown in FIG. 14, which comprises two anchors attached via a short length of flexible suture material 756. When anchor is embedded in the abdominal wall, the trailing anchor would engage with the inner facing surface of a mesh to provide secure fixation.

To use the device of FIGS. 11 to 13 the user:
a. Passes the device through a trocar until the distal tip of the device is in contact with the inner abdominal wall at the target location;
b. Compress the handle to deliver the anchor;
c. allow the handle to return to its non-compressed condition;
d. the device from the trocar;
e. Load another anchor into the tip of the device;
f. Repeat steps a to e to deploy the next anchor and load the next anchor until sufficient anchors have been deployed.

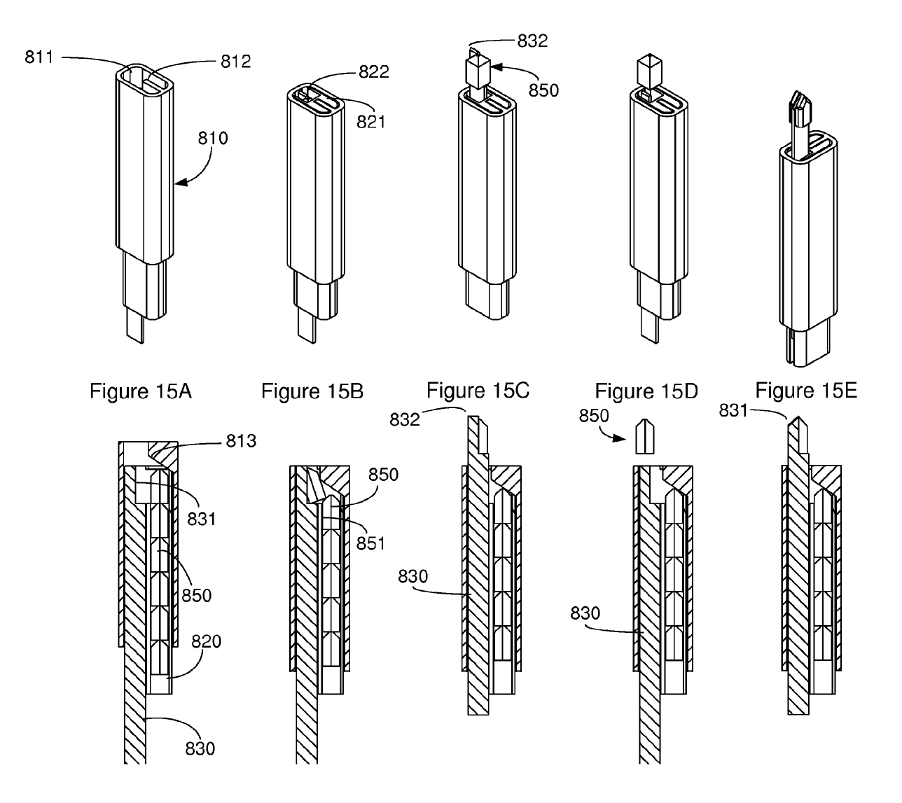

A further embodiment is illustrated in FIG. 15, specifically FIGS. 15A to 15E. An outer shaft 810 features a hollow interior 811 and a protrusion at the tip 812. A magazine 820 fill the interior space of the shaft apart from an opening 822. Also disposed within the shaft 810 is a needle 830. At the distal end of the needle is an anchor receiving portion 831. The anchors 850, as shown in the sectional view of FIG. 15A, are lined end to end in a channel in the magazine portion and are sprung forward (or towards the distal tip by a spring, not illustrated). The anchor 850 which is loaded in the magazine channel is forced forward by the spring force. The anchor contacts the underside of the anchor receiving portion 831 and is pushed sideways by the angled surface 813. A slot in the anchor 851 allows it to side load onto the needle receiving portion 831 as illustrated in FIG. 15B. As the next anchor is pushed forward it pushes proximal end of the leading anchor against the needle shaft. As the needle shaft 830 is advanced as shown in FIG. 15C, the leading edge 832 of the needle cuts through the tissue and allows the anchor to be deployed. FIG. 15D shows the anchor deployed and the needle shaft 830 retracted within the shaft. The needle edge 832 of FIGS. 15A to 15D is shown as a knife edge, but this edge 833 could also be angled to match the anchor geometry as illustrated in FIG. 15E. The device illustrated in FIGS. 15A to 15E shows an anchor which may be coupled to a suture (not shown).

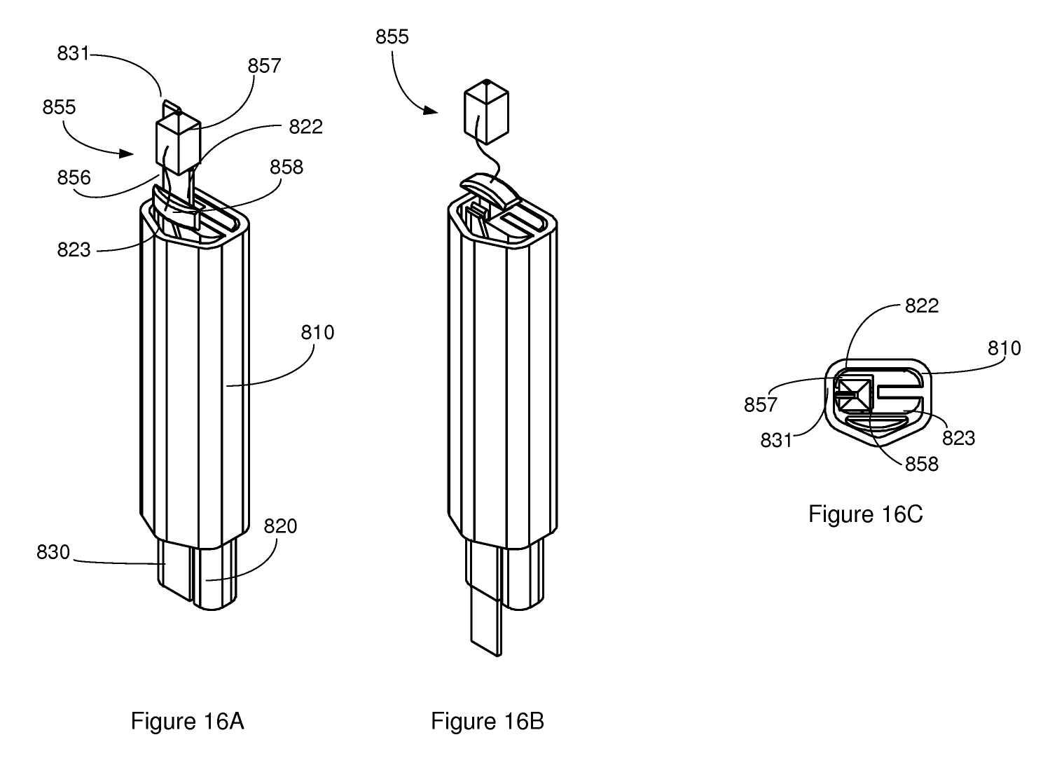

FIGS. 16A to 16C illustrate a device similar to that described in FIG. 15, but the anchor 855 is designed to have a proximal 858 and distal component 857, coupled via a tether 856. The distal portion 857 is actively driven into the tissue by the needle shaft 830 in a similar fashion to that described in FIG. 15. The proximal portions 858 are placed in the channel 813. When the distal portion is driven into tissue, the proximal portion is dragged out of the device via the tether 856 such that it follows the distal portion or first part of the anchor. The distal portion anchors in the tissue and the proximal portion abuts against the inner surface of a hernia mesh, to maintain mesh position against the abdominal wall. It will be appreciated that this arrangement separates the proximal and distal portions of the anchor during the passage through the device.

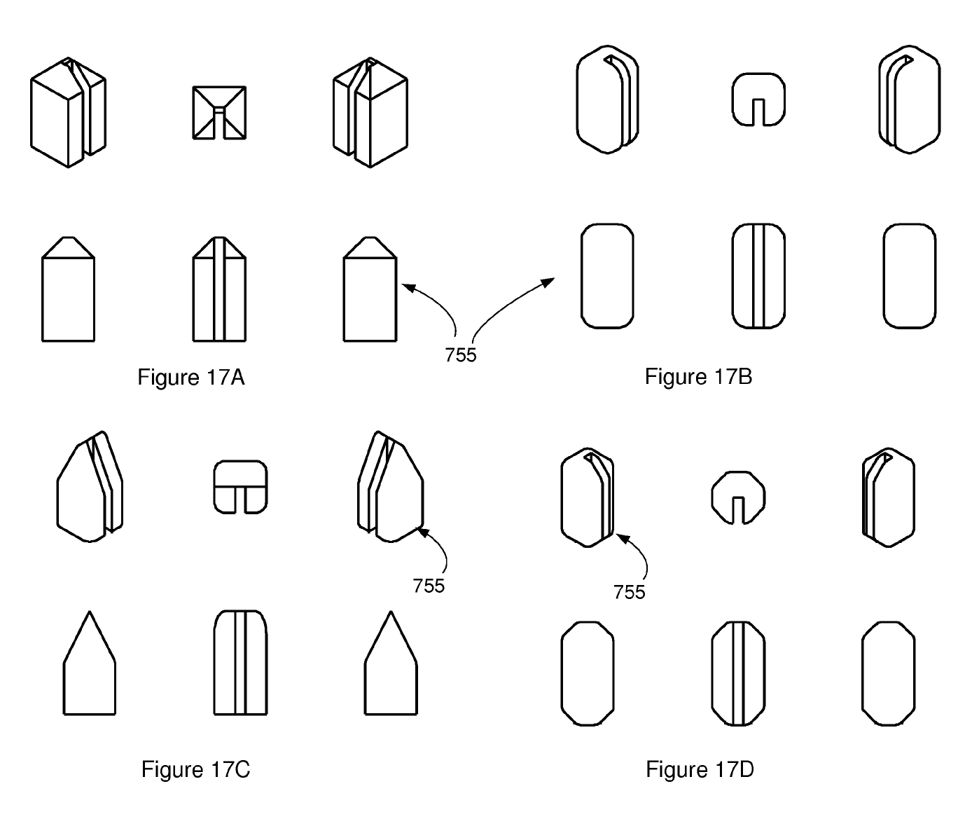

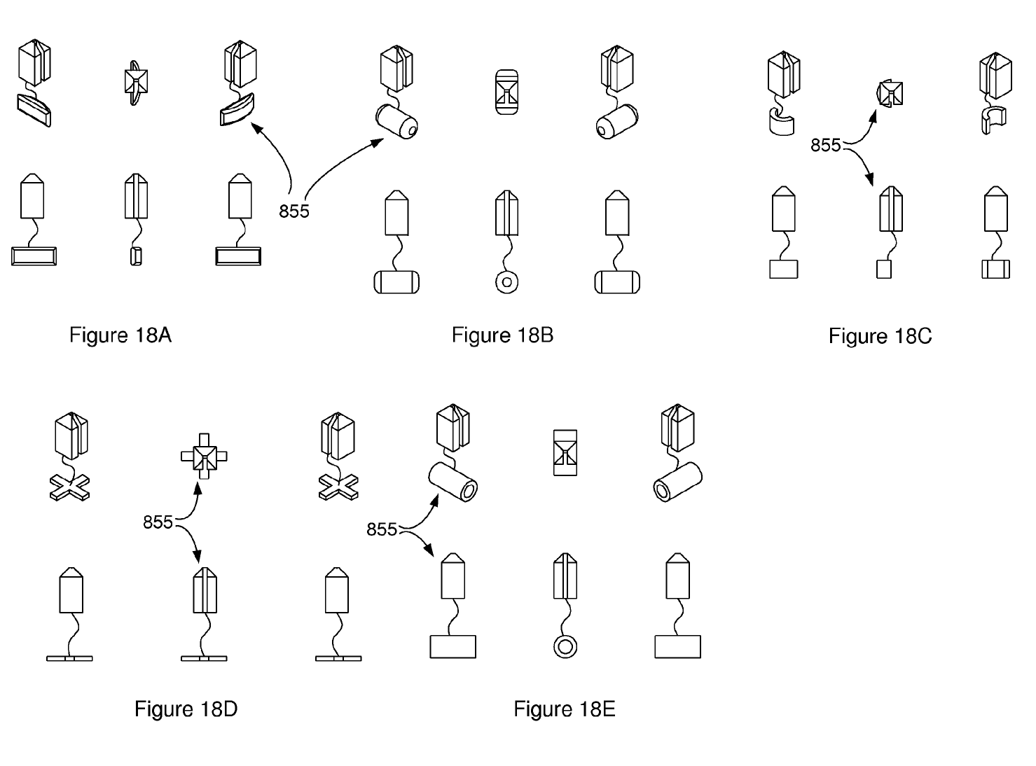

The geometry of the anchor illustrated in FIG. 15 is not intended to be limiting and other examples of suitable geometries that could be deployed are illustrated in FIG. 17A to 17D and in FIGS. 18A to 18E

The anchors 755 and 855 are shown here are for illustrative purposes and the geometries illustrated are not intended to be limiting. FIGS. 18A to 18E illustrate other examples of anchors with proximal 858 and distal portions 857 where the proximal and distal portions are different from that illustrated in FIG. 18A. While the examples of FIG. 17 are one piece components, it will be appreciated that these exemplify arrangements that could be used for the leading or first part of a two part anchor configuration.

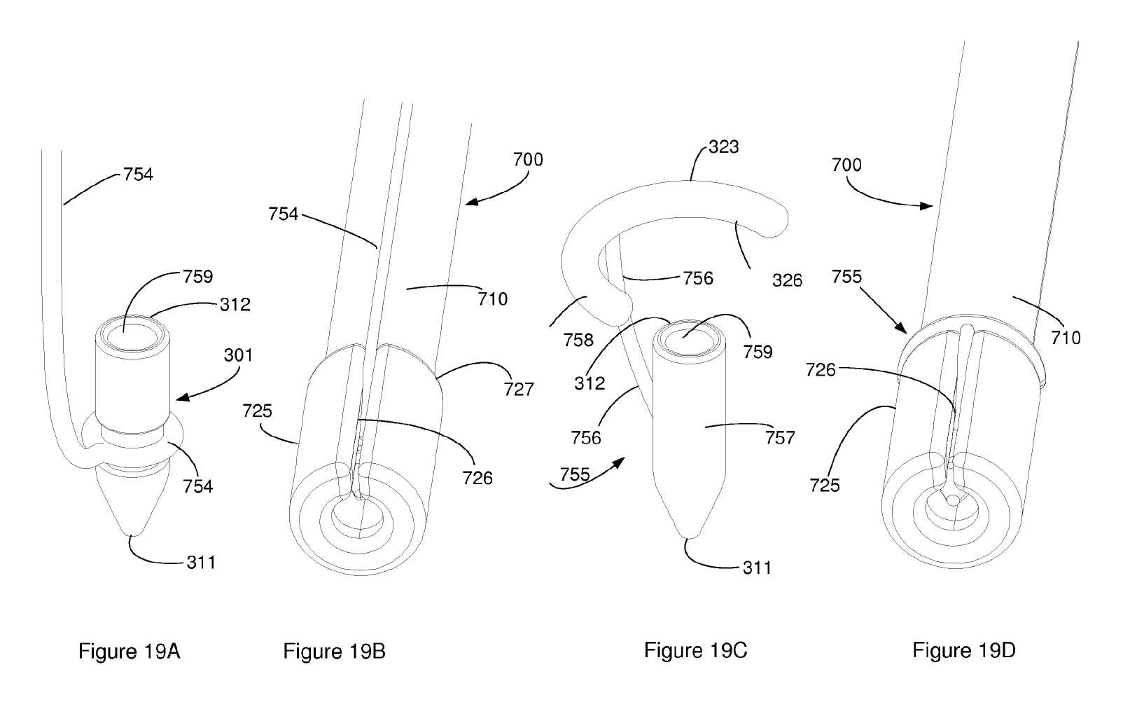

FIG. 19A to 19D, 20 and 21 show further embodiments of a device to deploy a single anchor. Unlike the device of FIGS. 11 to 13, in this device the core pusher wire that is used to deploy the anchor from the shaft 700 does not act as the cutting edge.

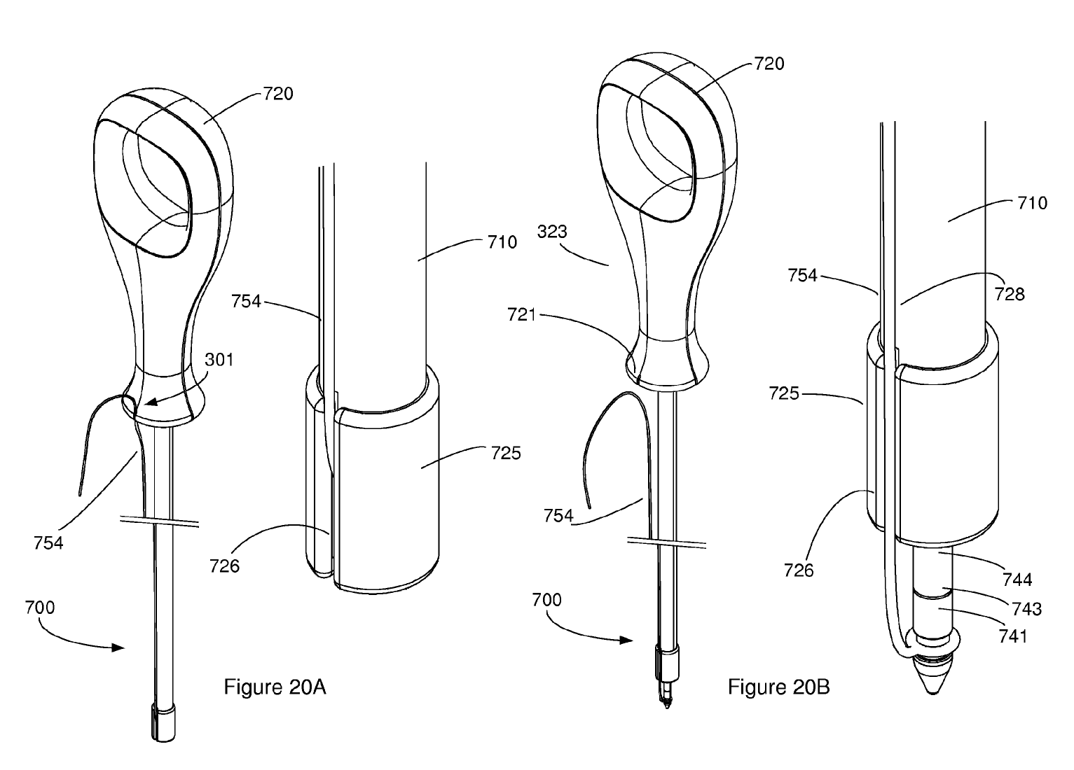

In FIG. 19A an anchor 301 is shown coupled to a suture 754. In this configuration the anchor is formed in a one-part construct comprising a leading edge portion 311 and a trailing edge portion 312. A lumen 759 is disposed on the trailing edge portion. The device 700 is show pre deployment in FIG. 20A. At the distal end of the device a larger diameter engagement portion 725 feature a slot 726. The larger diameter engagement portion 725 maximises the purchase on the abdominal wall or mesh. This engagement portion feature may be manufactured from a polymer and may be tacky or feature a textured surface to improve grip. Such a feature could be advantageously employed to prevent slippage of the device once a desire tack location is selected. The slot allows the suture to return to the handle. The handle 720 features a suture cleat 721, where the suture 754 can be pulled, ensuring that the anchor does not fall out of the distal tip of the device prematurely. FIG. 20B shows the device post deployment. The suture 754 has been released from the suture cleat. The transition between the small diameter portion 741 and the large diameter portion 744 results in a step 743. This step engages with the proximal end of the anchor, and drives the anchor forward as the device is being deployed. Alternately, the wire could be a single (smaller) diameter and engage with the anchor by abutting with the distal end of the lumen 759. The shaft 710 is connected proximally to a spring (not shown) which is housed in the handle. As the handle is pushed forward the shaft retracts, compressing the spring which exposes the anchor with the pusher wire inside.

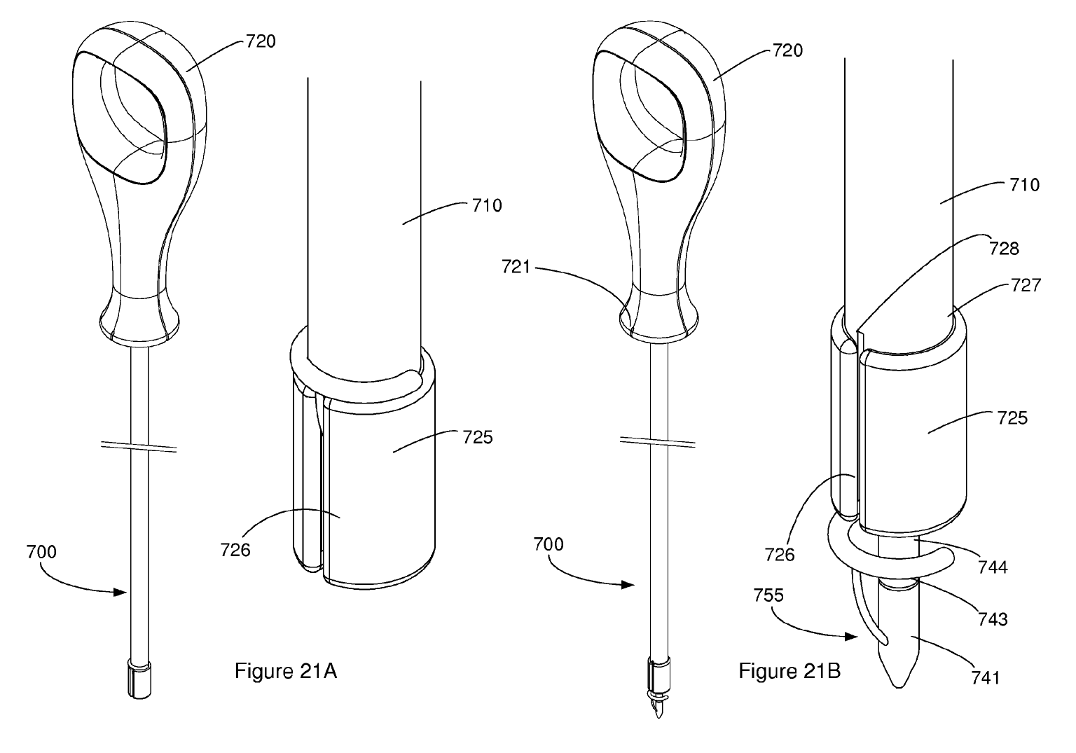

In FIG. 19C an anchor 755 is shown being formed from a two-part construct. The anchor comprises a proximal portion 758 and a distal portion 757, which are coupled together via an interconnect member 756 which allows relative movement between the first part and the second part. The anchor has a leading edge portion 311 and a trailing edge portion 312. A lumen 759 is disposed on the trailing edge portion. The device 700 is show pre deployment in FIG. 21A. At the distal end of the device a larger diameter portion 725 feature a slot 726. The larger diameter engagement portion 725 functions in a similar fashion to that described above and maximises the purchase on the abdominal wall or mesh. This feature may be manufactured from a polymer and may be tacky or feature a textured surface to improve grip. Such a feature could be advantageously employed to prevent slippage of the device once a desire tack location is selected.

The slot 726 allows the interconnect member portion of the anchor to pass towards the proximal end of the raised diameter portion. The proximal portion of the anchor features a curved inner portion 326. This curved inner portion clips over the shaft 710, and the interconnect member is tensioned such that the proximal portion is held in place by the step created by the proximal end of the raised diameter portion 725 and the shaft 710. FIG. 21B shows the device post deployment. The transition between the small diameter portion 741 and the large diameter portion 744 results in a step 743. This step engages with the proximal end of the anchor, and drives the anchor forward as the device is being deployed.

Alternately, a drive wire could be provided. In such a configured the drive wire would engage with the anchor by abutting with the distal end of the lumen 759. The shaft 710 could be connected proximally to a spring (not shown) which is housed in the handle. When the handle 720 is pushed forward the shaft retracts, compressing the spring which exposes the anchor with the pusher wire inside. When the handle is released, the spring pushes shaft 710 forward to cover the end of the exposed wire. As the anchor is deployed, the proximal anchor portion 758 is released from step 727, due to forward motion of the proximal portion of the anchor transmitted through the flexible member.

FIG. 21A shows the second part of the anchor dimensioned to sit on the outer surface 710 of the handle shaft. In another variant (not shown) it could be configured to sit on the outer surface of the larger diameter engagement portion 725. In another configuration (again not shown), the second part of the anchor could be sized to sit on the outer surface of the first part 741 of the anchor or the correspondingly the outer surface of the tool 744. In either of these latter variants, it is desirable that the second part be accommodated within a volume defined by the larger diameter engagement portion 725, and as such the surface area of the second part will be constrained by the diameter of larger diameter engagement portion 725.

The larger diameter engagement portion 725 described above is provided as an integrally formed element. In a variant, not shown, the engagement portion could be provided as a removable element which is received on the shaft 710. The engagement portion 725 could provide a housing for one or more anchors which could be pre-loaded into the engagement portion. To facilitate the loading of multiple anchors, the engagement portion may include a trailing substantially cylindrical surface that would sit onto the outer surface of the shaft. The join of the flexible leading surface of the engagement portion to the trailing cylindrical surface could provide a step against which multiple individual distal portions 757 would be stacked. Each of these distal portions would be coupled to a proximal portion that is located within the volume defined by the shaft. Extraction of a proximal portion would effect a pulling of the corresponding distal portion over the step and away from the device. It would also effect a corresponding movement of the distal portion of the next anchor upto the step until it is deployed.

Advantages of the devices described above from FIGS. 19A to 19D, 20 and 21 include the fact that the wire pusher wire does not feature a sharp tip, and could be reloaded by pushing the anchor into the tip of the device and affixing the suture in the suture cleat for the device of FIG. 19A, or clipping the proximal anchor portion onto the shaft for the device of FIG. 19C.

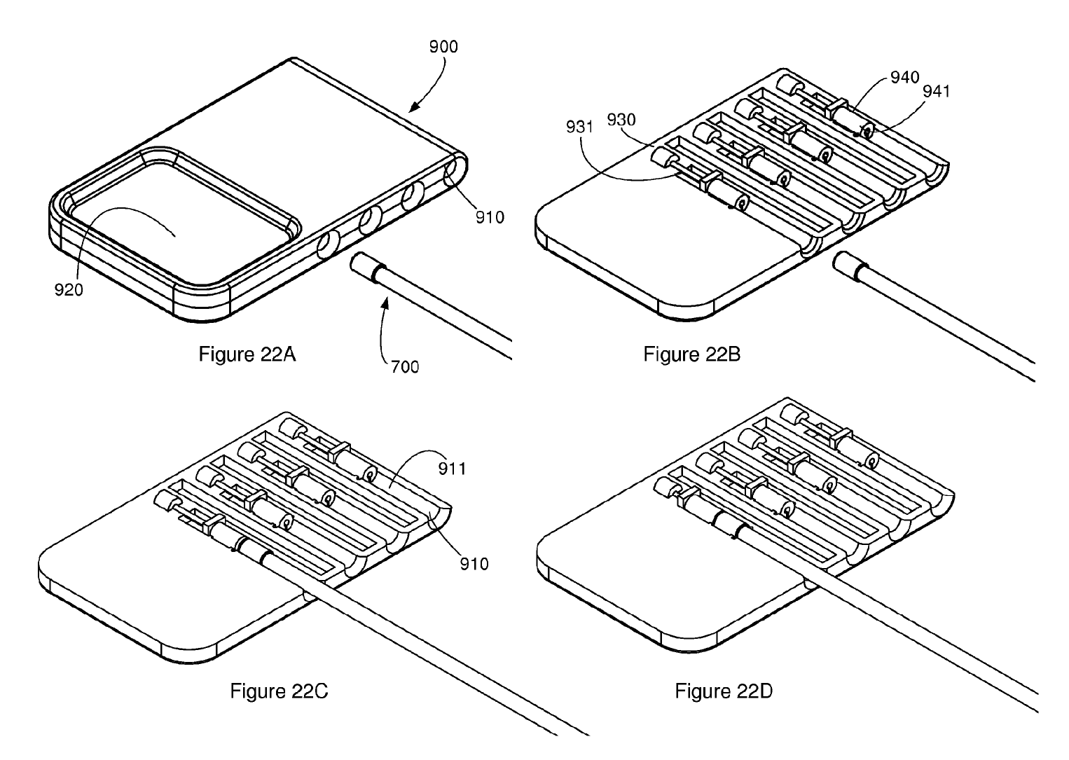

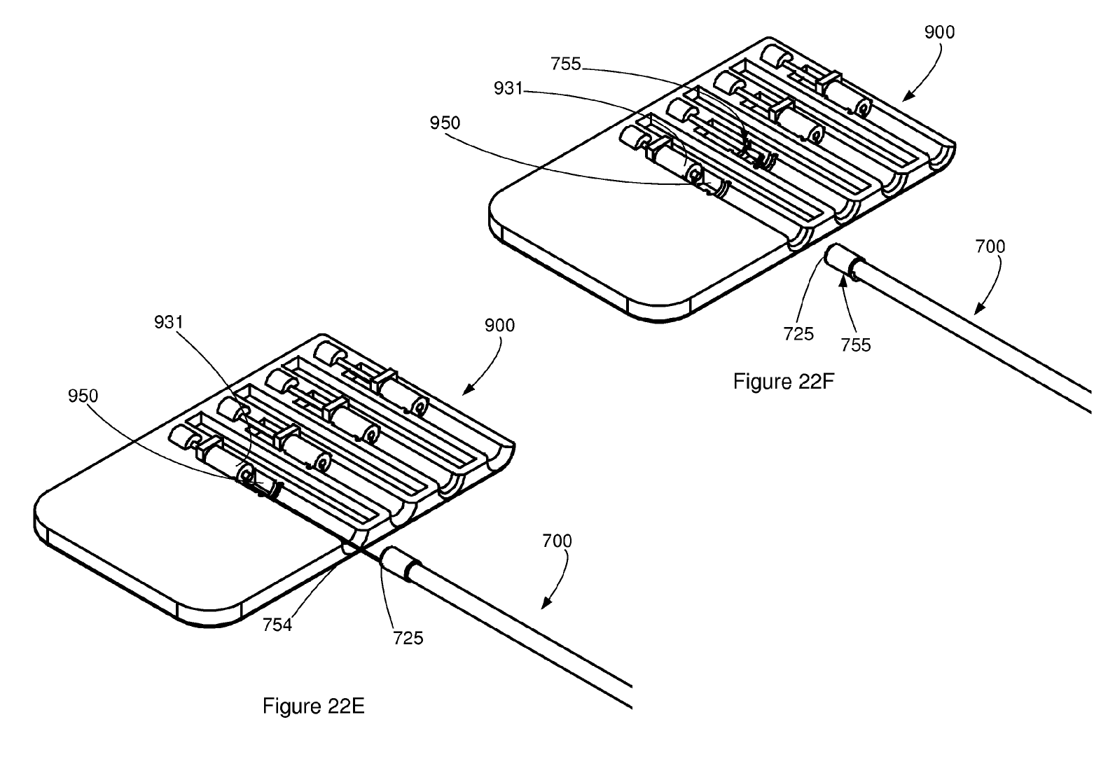

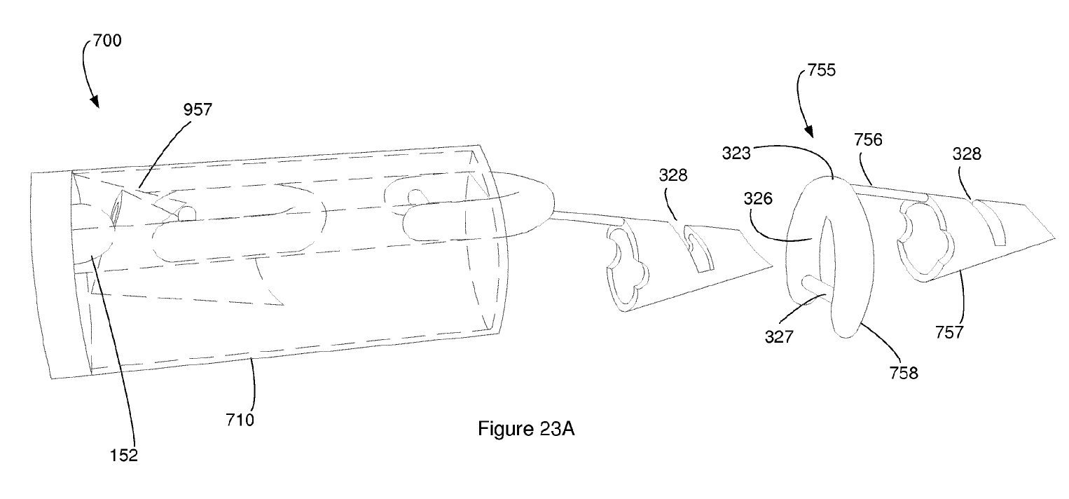

Another solution to reloading the device is presented as FIGS. 22A to 22F. Here a cartridge 900 is provided which in this exemplary configuration features four preloaded anchors in lumens 910. A depression 920 is provided as a grip on an outer surface of the cartridge, but is not intended to limit the present teaching to such a configuration, as the geometry of this feature could be varied, textured or raised to improve the grip. The device 700 is presented to the lumen 910 and passed into the lumen as illustrated in a cutaway view FIG. 22C. The anchor is loaded in a lumen 941 of a slideable portion 940 of the cartridge 900. The cartridge is also features a fixed pusher portion 930, which features a pusher rod 931, which is sized to fit into the lumen 941 of the slideable portion 940. The device 700 is pushed into the cartridge until it meet a stop. As the device 700 advances through the lumen 910 it pushes the slideable portion 940 forward. The anchor (not shown) remains stationary, as it is in abutment with the pusher rod 931. The device 700 passes over the anchor and picks it up due to a slight interference between the outer profile of the anchor and the inner profile of the tip of the device. As the device is removed from the cartridge the suture is dispensed from the cartridge for the device of FIG. 19A. For the device of FIG. 19C the proximal portion of the anchor would be in the recess 950, and would be picked up by the device once the raised diameter portion passes distal to that point. As the device is retracted from the cartridge, the larger diameter portion 725 prevents the clip from detaching from the device as the larger diameter portion is sized to just fit in the lumen 910.

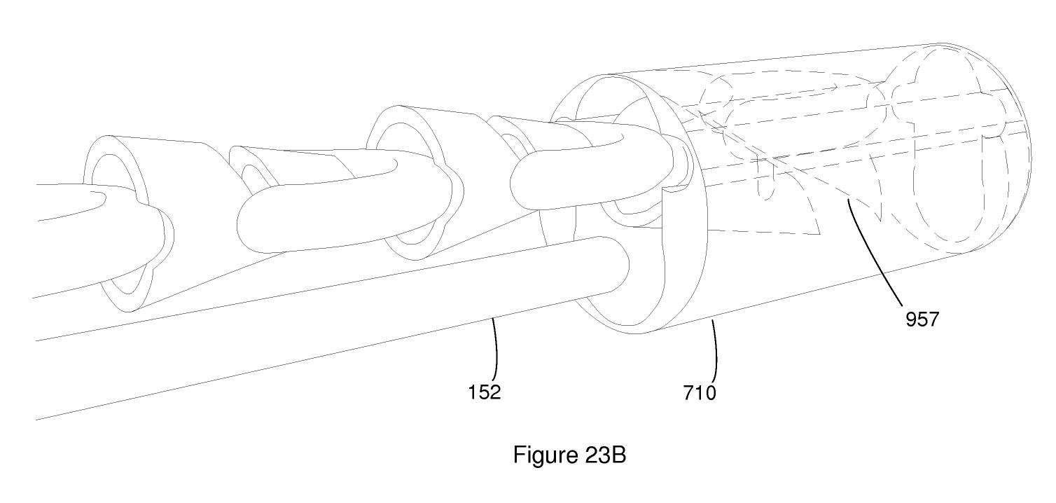

FIGS. 23A and 23B show another configuration of an anchor 755 which again comprises a first part defining a proximal portion 758 and a second part defining a distal portion 757 coupled together by an interconnect member 756 which may be flexible in nature. In this variation the proximal member features a crossbar 327 which seats into the slot 328 of the distal portion when the anchor is ion the shaft of the delivery device. The tip of the device 700 features two openings, which are uniquely sized to accommodate each portion of the anchor, and not the other. As the pusher 152 engages with the distal portion of the anchor 757, an ramp 957 on the inside of the device shaft 710 deflects the distal portion of the anchor downwards towards its exit channel, this in turn disengages the cross bar and allow both components to be ejected from the device by the pusher, with the distal portion being actively driven by the pusher, and the proximal portion trailing as it is pulled along by the flexible member. This method of engaging both portions also acts as a safety mechanism against premature deployment, as the anchors can only exit the shaft when the pusher pushes the distal portion of the anchor forward. It will be appreciated that a two part anchor per this configuration could be used independently of other anchors or elements that are described in this specification. Such an anchor comprises a first part and a second part coupled together through an interconnect member, wherein the first part of a first anchor is configured to interlock with a second part of a second anchor to restrict deployment of the second anchor can be advantageously used in any configuration where a plurality of anchors are arranged for sequential deployment. To effect controlled deployment, the present teaching provides in a first action a separation of the interlock arrangement and as a second subsequent action the discharge of the second anchor away from the first anchor.

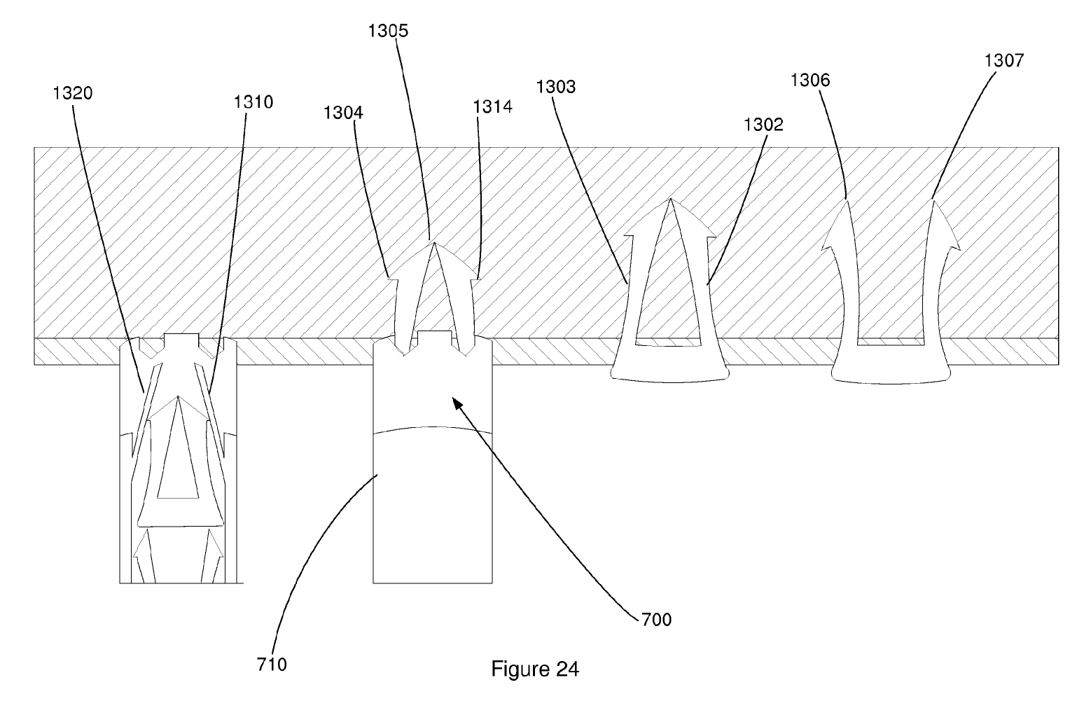

FIG. 24 illustrates yet another type of anchor that may be used within the context of the present teaching. The anchor 1301 is a single piece component and comprises two wing portions 1302 and 1303, each of which have a sharp tip 1306 and 1307. As the anchor 1301 is being delivered from the tip of the device 700, a pair of leaf springs 1310 and 1320 compress the wings 1302 and 1303 together to create a single tissue piercing point 1305. It will be appreciated that other arrangements could be used to bias the first and second wings together to the single tissue piercing point 1305. As the anchor nears the end of its deployment cycle the force of the leaf springs tapers off, allowing the wings to spring outwards. Additional fixation is achieved by the addition of barbs 1304 and 1314, which prevent the anchor from easily pulling out of the abdominal wall. Such an anchor comprising two leading wing portions which are movable relative to one another to create a tissue piercing point during deployment and which subsequently separate from one another to effect retention of the deployed anchor within the pierced tissue could be used in any one of a number of different anchor arrangement including one and two part anchors such as those described elsewhere in the present specification.

The suture may be a braided suture made from a bioabsorbable polymer such as PGA for example. For fascial layer closure a USP size 0 suture is preferred. This material is ideally suited to an application where the suture maintains approximately 50% of its strength after two weeks. However it will be appreciated that the suture material may be changed depending on strength or mass loss requirements of the specific application. The anchor could be moulded from a bio-absorbable material. The material in a preferred embodiment is PLGA but could be made in any ratios of the following materials PGA, PLLA, PDLGA, PLDLA.

A further modification could be made to any of the devices shown, which would be the provision of a fibre optic cable running to the distal tip of the device. The fibre optic could emit a beam of light at the tip of the device, to assist the surgeon in selecting the desired location for placement of the anchor.

The multi anchor devices illustrated here are single part devices, however a person skilled in the art will appreciate that the proximal portion of the device that advances the pusher and the distal portion of the device that houses the anchors, could be supplied as separate components which are combined to form a single device. The advantage of this approach would be that the anchors could be supplied in different cartridge sizes, for example 10, 20 or 30 anchors, which could eliminate waste and accommodate surgeon preference, as some surgeons prefer to use more anchors than others. Additionally, the handle could be manufactured form materials that are autoclavable, meaning this portion of the device is reusable, and only the cartridge is supplied as a consumable. Where the anchors are provided as two part anchors with each of the two parts coupled to one another by an interconnect, the length of the interconnect will determine how the anchor will ultimately be deployed. Different cartridges could be configured to be used with different anchor types and in this way a surgeon could select the appropriate anchor for a particular procedure by simply changing the cartridge.

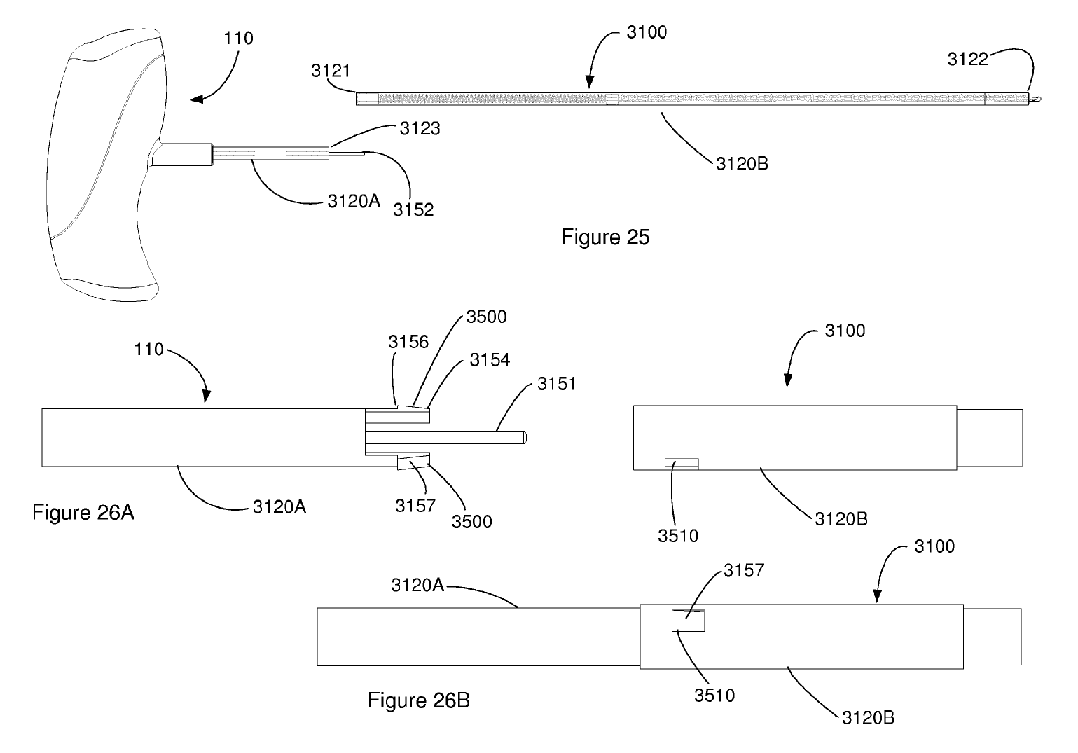

An example of such a device is shown in FIG. 25 where a handle portion 110 is supplied separate to the cartridge portion 3100. The distal end 3123 of the shaft portion of the handle is joined to the proximal end of the cartridge portion 3121. Various mechanisms may be employed to achieve this union and the examples illustrated here are not thought to be limiting. FIG. 26A shows some of the detail of the connecting mechanism in one embodiment, whereby the end of the handle portion has a pair of wings 3500. The wings are tapered outwards from the distal end 3154 to the proximal end 3156, such that a barb type feature is created. When the cartridge portion 3100 is joined with the handle portion 110, the wings 3500 slide inside the cartridge shaft 3120 until the wings click into the openings in the shaft 3510 as illustrated in FIG. 26B. The shafts may be keyed to align the wings 3500 with the openings 3510. A simple tool could be provided which is passed onto the shaft whereby a feature on the tool would interact with the surface of the wing 3157 to depress the wings, thereby allow the cartridge to be decoupled. Alternately, such a tool could be mounted on the shaft of the cartridge portion.

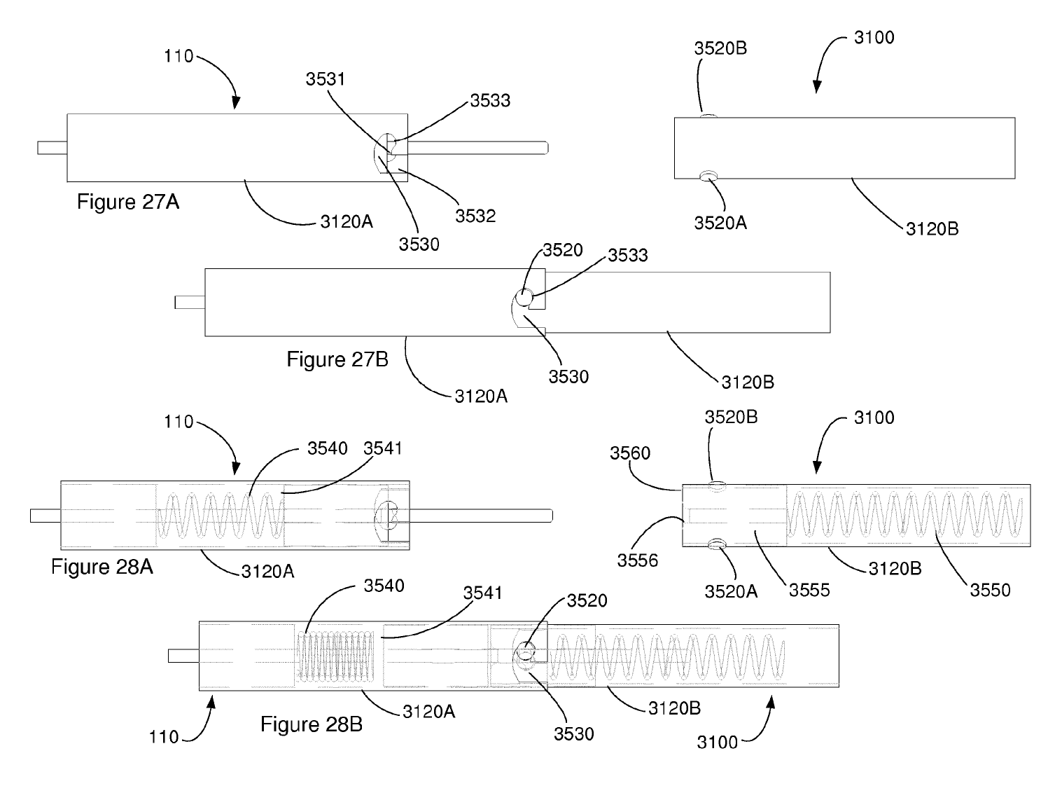

Another embodiment of a coupling mechanism is illustrated in FIG. 27. The cartridge portion 3100 features a pair of protrusions 3520. These protrusions couple with two slots 3530. As the two portions are coupled the protrusion pass into a portion 3532 of the slot 3530 which is substantially parallel to the shaft 3120A. The user then rotates the cartridge portion clockwise to lock it in place, with the protrusion 3531, preventing rotation of the cartridge. In FIG. 28A a spring 3540 maintains the forward position of a slideable portion 3541. When the handle portion and cartridge are joined, the proximal end of the cartridge 3560 depresses the slideable portion 3541. The spring 3540 is compressed, and when the cartridge is rotated, the spring extends slightly to maintain the protrusions 3520 against the surface 3533.

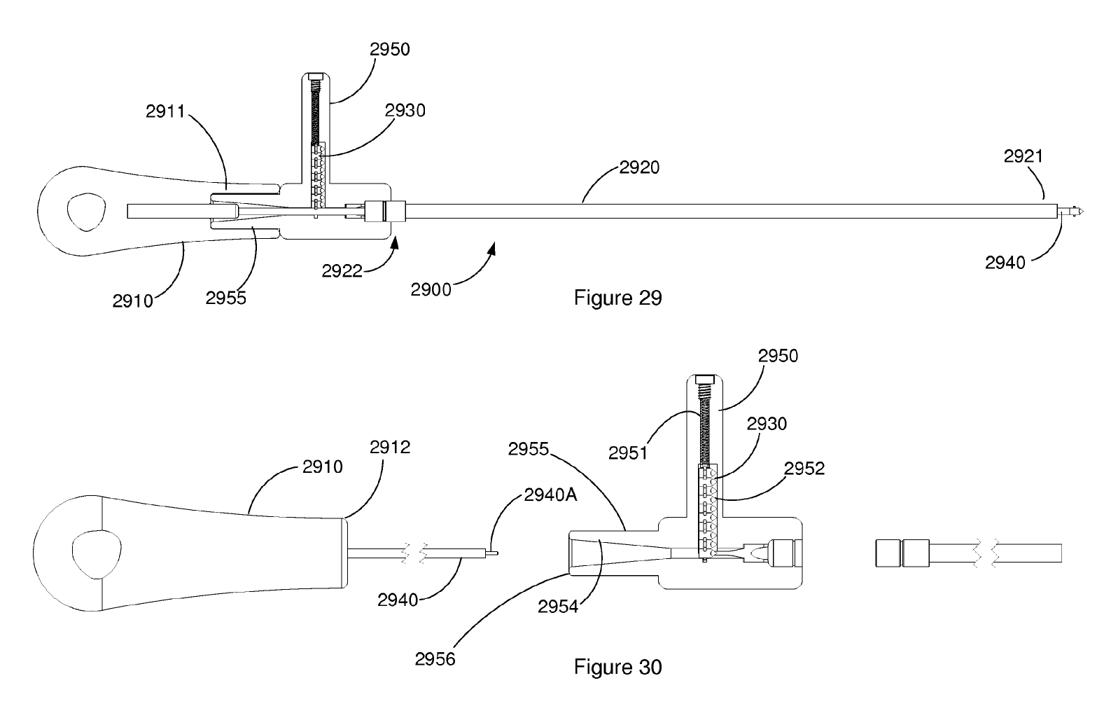

FIGS. 29 to 33 show another example of an anchor delivery or tacking device that may be used within the context of the present teaching. In this configuration and similar to previously described configurations, the device 2900 comprises a handle 2910 coupled to a shaft 2920. The shaft 2920 comprises a handle distal end 2921 of the shaft and a handle proximal end 2922 of the shaft. The shaft in this arrangement is hollow and dimensioned to accommodate at least a portion of an anchor 2930 therein.

The device 2900 comprises an actuator 2940, in this arrangement a driver configured to effect a displacement of the anchor away from the shaft. Similarly to the previous configurations the device is configured to deploy two part anchors, a first part being dimensioned to positively engage with the actuator during a delivery of the anchor out of and away from the shaft, and a second part configured to follow the first part during the delivery such that the first part is displaced away from the shaft prior to an exit of the second part away from the shaft.

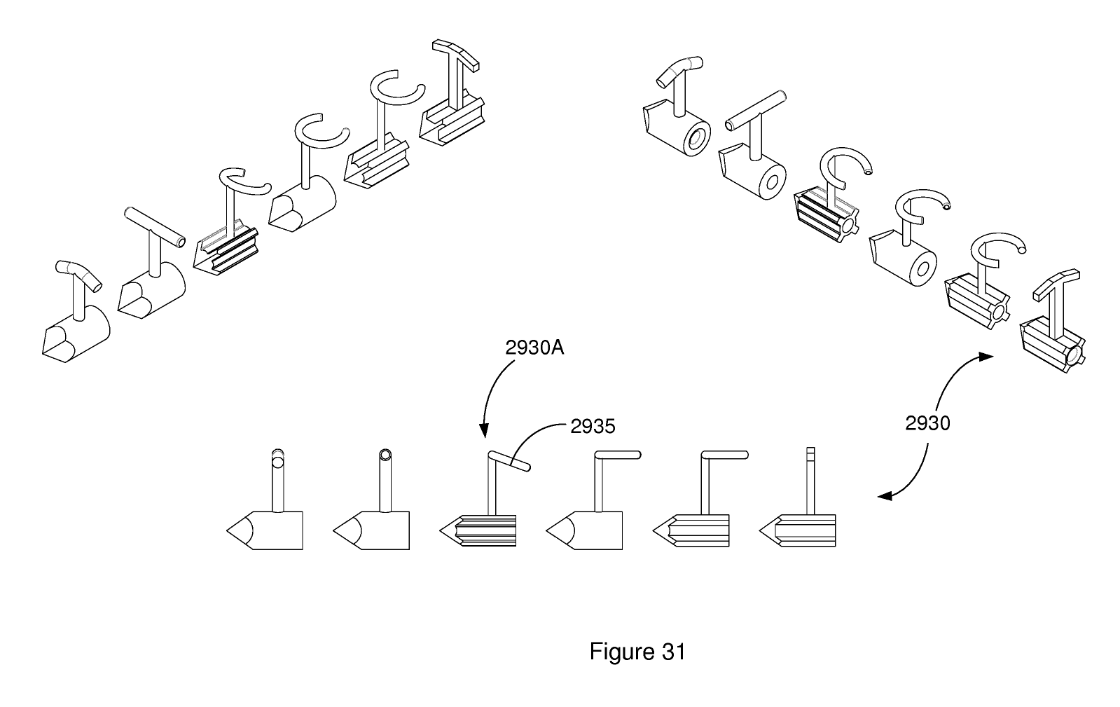

As shown in FIGS. 30, 32 and 33 the actuator 2940 and first part of the anchor 2930 are configured to engage with one another during a delivery of the anchor out of the shaft 2920. In this configuration, the actuator driver 2930 is coupled to the handle 2910 and the two elements are then moveable relative to the shaft 2920. The actuator 2940 has a length such that on completion of the delivery action, a tip portion of the actuator projects beyond the handle distal end portion 2921 of the shaft 2920.

The actuator 2940 comprises an anchor engagement portion 2940A which is receivable within at least a portion of the first part of the anchor—such as for example a lumen provided in a proximal portion of the first part of the anchor. In this configuration the outer diameter of the anchor engagement portion would be less than the inner diameter of a lumen or receiver formed in the anchor such that driver is receivable within the anchor. In another configuration the dimensions could be reversed such that the anchor is receivable within at least a portion of the actuator.

In this configuration the device 2900 is configured to receive a cartridge 2950, the cartridge comprising a plurality of anchors 2930. The anchors are spring loaded through use of a biasing member 2951 which positively maintains the individual anchors within a delivery channel 2952 of the cartridge 2950. In this configuration the delivery channel 2952 is orientated so as to be substantially transverse to a longitudinal axis of the overall device.

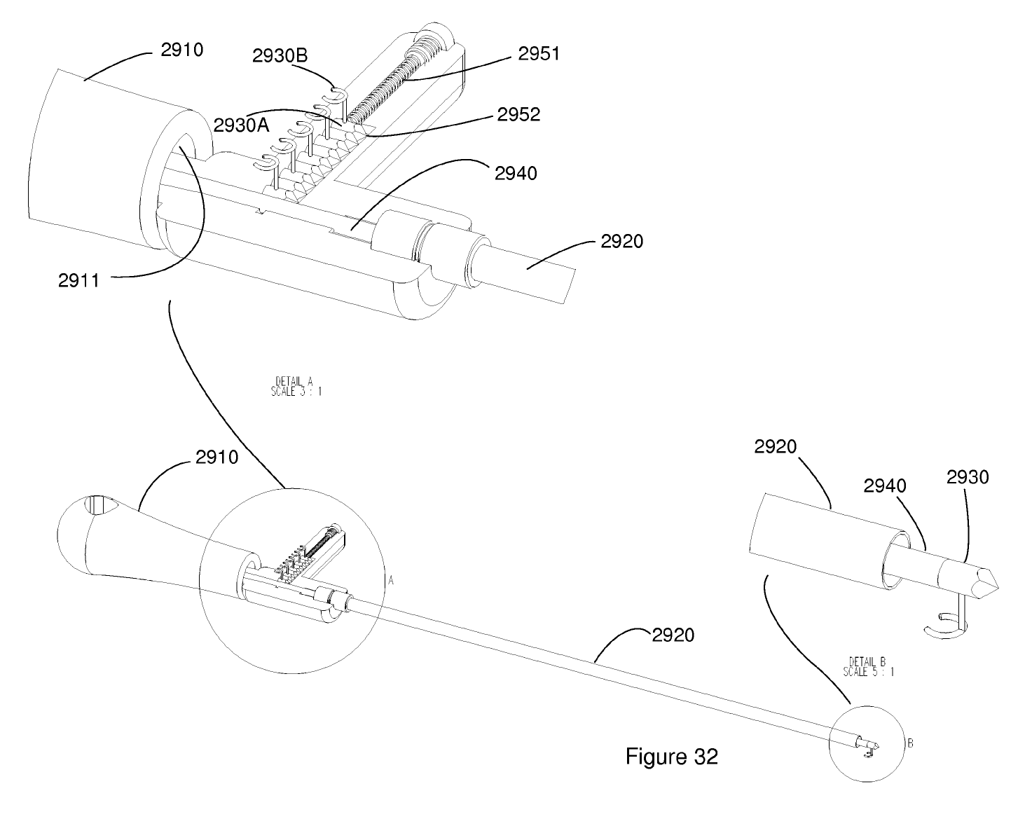

As shown in FIG. 32, each of the individual anchors 2930 are arranged such that respective first parts 2930A of the anchors are aligned within the delivery channel 2952. They are disposed therein such that a leading or distal end of the first part of the anchors are orientated in the same direction as the shaft 2920. The actuator is configured to selectively displace individual anchors from a received cartridge, such that a first anchor is delivered from the shaft prior to engagement of a second anchor with the actuator. Each of the anchors are delivered into the shaft 2920 and will be pushed through the shaft by the actuator. The orientation of the anchors within the delivery channel 2952 ensures that when they are displaced out of and away from the device, the distal end of the first part 2930A will exit the device first. The second part 2930B will follow this first part and will be displaced away from the shaft in response to the initial exit of the first part.

In this configuration each of the shaft 2920 and the handle 2910 are mateable with the received cartridge 2950, the cartridge operably being provided between each of the handle and the shaft. On engagement of the cartridge with each of the handle and the shaft, the cartridge is configured to positively bias anchors into engagement with the actuator. To facilitate ease of placement of the actuator 2940 i into the cartridge 2950 a tapered entry 2954 is provided in the cartridge. The cartridge also features an extended proximal section 2955, which terminates proximally at the radiused edge 2956 and distally at the step 2957. This portion of the cartridge is sized to be received into the handle recess 2911. This is advantageous as it shortens the overall length of the device, making the device less cumbersome to handle. In addition this lower profile zone acts as a warning to the user that the anchor is about to emerge from the tip of the device as the leading edge of the handle 2912 begins to engage with the proximal end of the cartridge 2956. The extended profile 2955 also provides an overlapping section, so that as the user retract the actuator 2940, this over lapping section reduces the likelihood of the user disengaging from the cartridge. In another modification, not shown, the actuator could be marked, for example through use of a colour to provide a visual identifier or warning to the user that they were close to disengaging it from the cartridge. Prior to the engagement with the actuator, the anchors within the cartridge are biased in a direction substantially transverse to a longitudinal axis of the device. By providing the handle and shaft separately to the cartridge it is possible to provide a kit of parts whereby the same handle and shaft may be used with different cartridges which may be made and sold separately. As the actuator and the shaft are actively provided into the abdominal cavity these will require re-sterilization between uses. The material used for fabrication of these components may be optimised for such re-sterilization whereas the cartridge which is not intended to be reusable and will be manufactured from appropriate materials which may be injection molded.

Referring to the two part anchor configurations described herein and illustrated in for example FIGS. 18 and 31, it will be appreciated that the length of the interconnect or connector between the first anchor part and the second anchor part can be varied. This is useful when for example a hernia mesh is being fixed in an obese patient with a significant layer of pre-peritoneal fat. In such instances current tacks in lengths of up to 10 mm are mainly anchored in fat. If the connector is, for example, extended to 15 mm, then the first part of the anchor may be delivered into and anchor in the fascia muscle or suitable deeper tissue layers, which would provide a more secure attachment of the mesh. This longer embodiment could also be used as an alternative to trans-fascial sutures, commonly used to fix the corners of the mesh.

A further modification is shown in Anchor 2930A of FIG. 31, where the second anchor part is anchor angled such that on delivery into the final configuration the surface 2935 is in contact with the mesh and parallel to the inner abdominal wall. The angle may also be configured so that it is sprung against the mesh ensuring optimal fixation. The feature would also prevent the second part extending into the abdomen ensure a lower profile.

When anchors with extended interconnects are used with the device of FIG. 29, specific pushers for longer tail lengths could be used as the actuator element 2940 is interchangeable with the handle 2910. Alternatively, the length of the shaft 2920 could varied such that alonger shaft is provided with the longer tacks, to enable sufficient penetration of the actuator 2940.

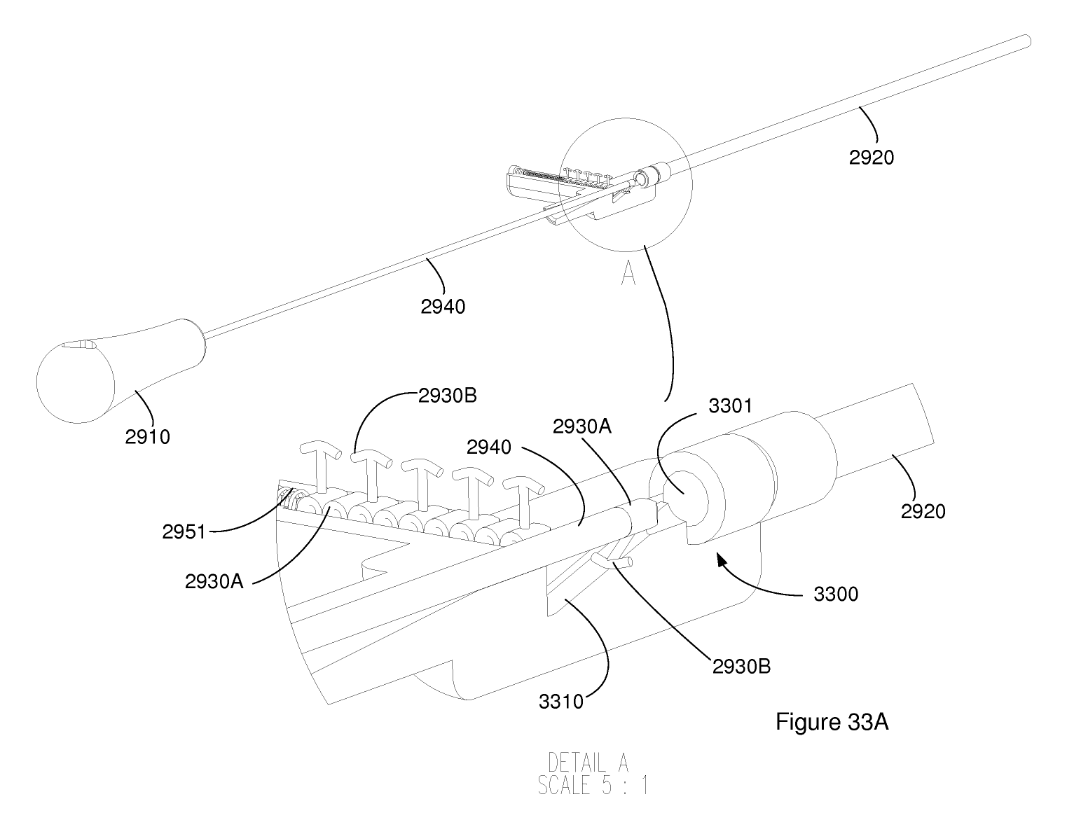

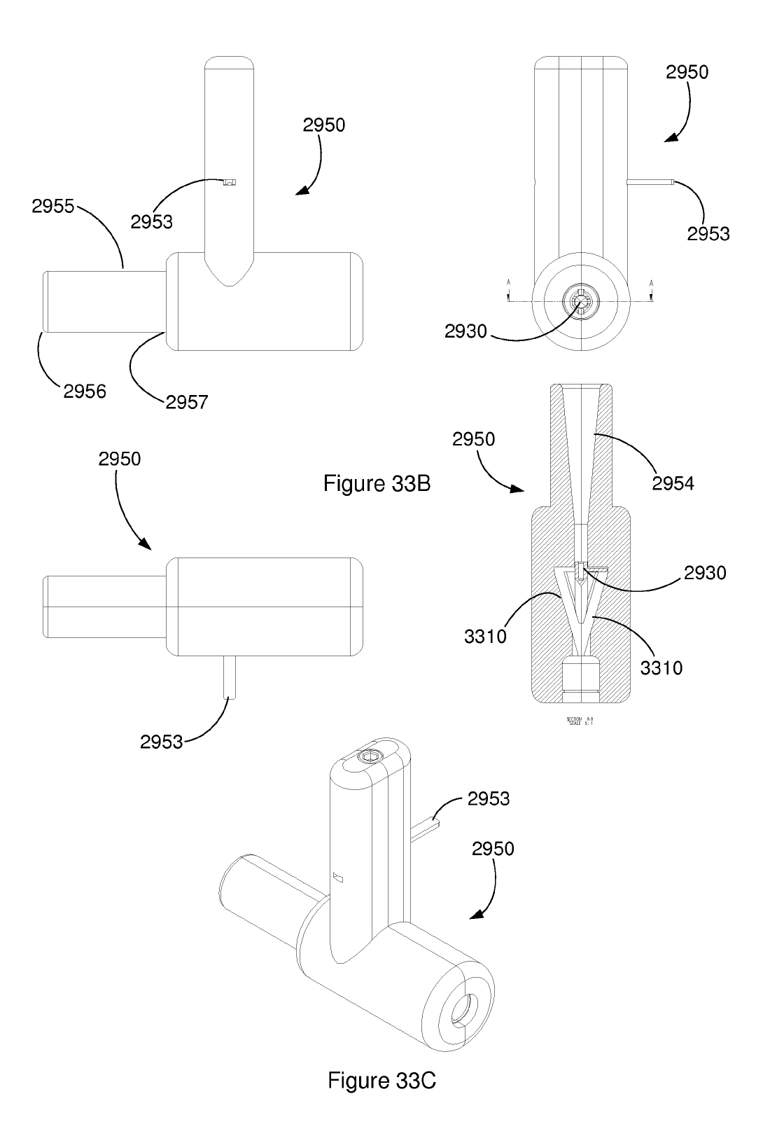

As shown in FIG. 33A, the delivery channel is arranged to terminate at an anchor engagement region 3300. When the actuator is located within the shaft 2920 it blocks a delivery of anchors from their delivery channel into the anchor engagement region 3300. On withdrawal, access to the anchor engagement portion is provided and an anchor can be delivered into the anchor engagement region. As discussed before, a proximal part of the first part 2930A engages with the actuator. The second part engages with a ramp 3310 which is provided to ensure that that the second part 2930B does not interfere with the introduction of the first part 2930A into the shaft lumen 3301. In the schematic of FIG. 33A, only one ramp is shown but it will be appreciated that this is for ease of viewing and a first and second ramp are provided—per the sectional view of FIG. 33C- to selectively engage with individual ones of the second parts which are located within the delivery channel 180 degrees out of phase.

As is shown in the detailed view of FIG. 33B, the cartridge which may be provided as a separate component with a plurality of anchors pre-located therein. The anchors are spring loaded through use of the biasing member 2951 but to avoid direct action of the biasing member during transit, a locking member 2953 is provided between the biasing member 2951 and the anchors. This can be removed out of location—per the arrangement of FIG. 33B to provide contact between the two.

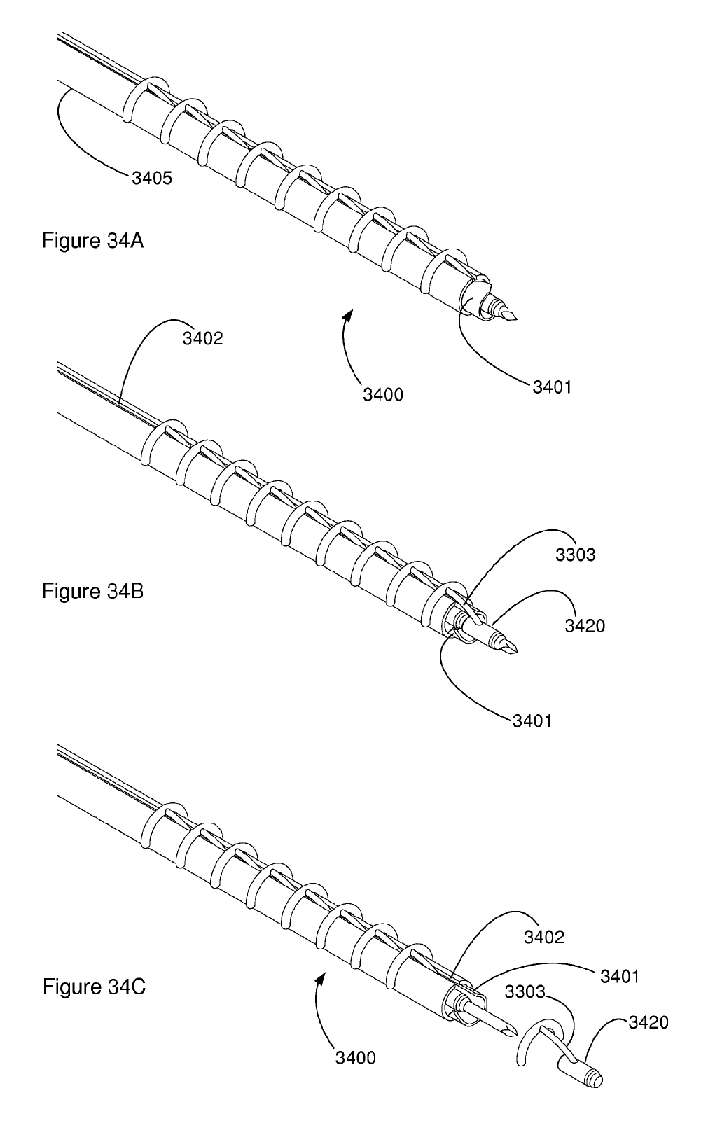

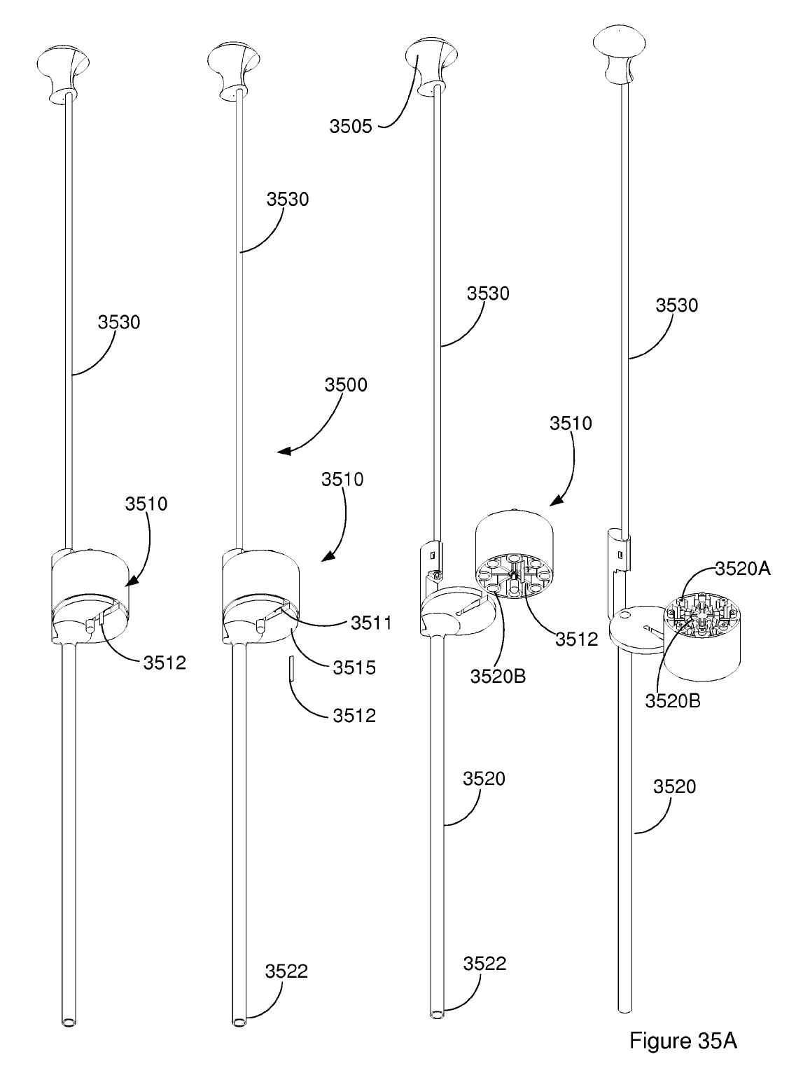

FIG. 34 (FIGS. 34A, 34B & 34C) shows another arrangement of a delivery device 3400. The device is similar in operation to that described with reference to FIG. 19, in that a first part of the anchor is disposed within a shaft 3401 of the device and a second part is disposed on an outer surface of the shaft 3405. In this configuration the shaft 3401 is a rotatable shaft that is connected back the handle (not shown) in its resting configuration. In this way, the shaft blocks or prevents introduction of a subsequent tack or anchor into the shaft, by blocking the interconnect member 3303 from exiting the interconnect channel 3402. As shown in FIG. 34B, when rotated, access is provided to the next anchor of the series of anchors that require delivery. When rotated the next anchor can be deployed. This shaft 3401 could be sprung in the previous configuration. Depressing a trigger could rotate it to release an anchor and a cam could cause it to adopt the sprung configuration. As shown in FIG. 34C, an anchor 3420, which again is provided in a two part form, may also be biased to advance forward, either directly or by springing a pusher rod that engages with the proximal end of the anchor. In this way it is possible to ensure that the most distal anchor advances until the rotatable shaft obstruction. This may be used to selectively deliver individual ones of a plurality of anchors in a controlled fashion.

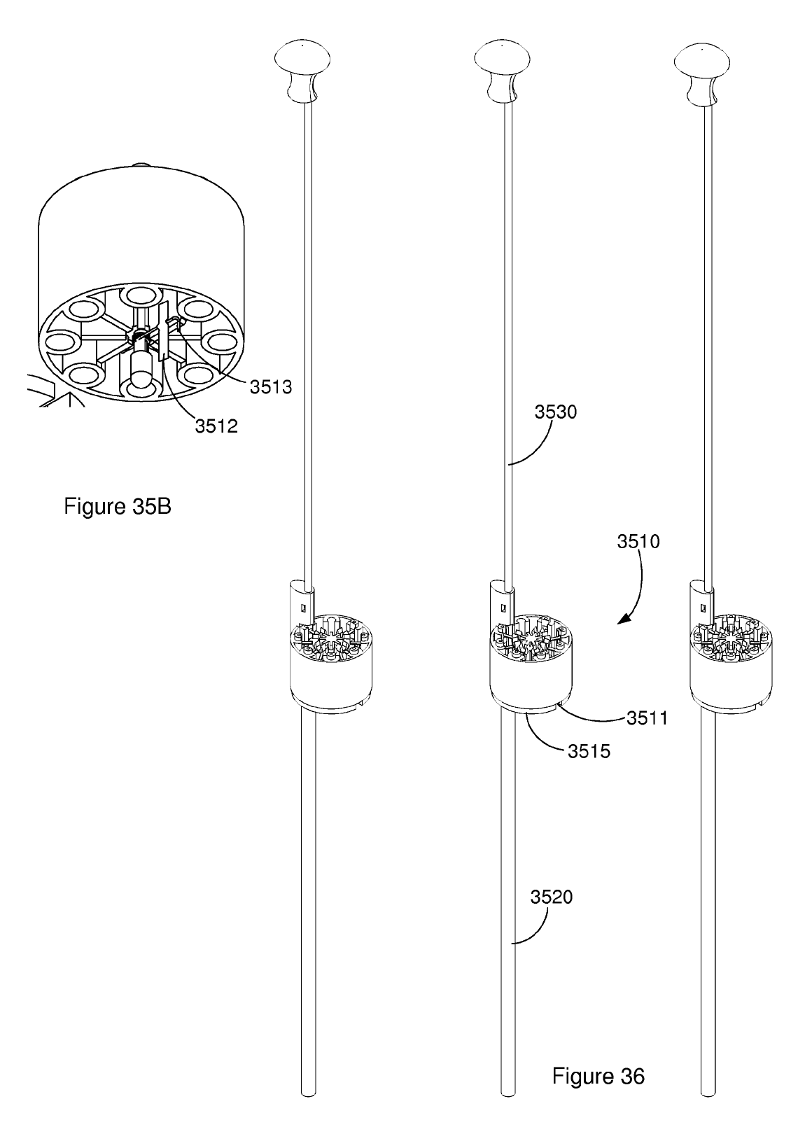

FIG. 35A shows a further configuration of an anchor delivery system 3500 in accordance with the present teaching. Similarly to previously described arrangements this system is configured to allow a user selectively and controllably deliver individual ones of a plurality of anchors. As was described before, the system comprises a handle 3505 and a shaft 3520. The shaft 3520 in this arrangement is hollow and is used to facilitate the delivery of an anchor out of a port provided at a handle distal end 3522 of the shaft. The anchors (not shown) are directed through the lumen of the shaft using an actuator 3530, which is coupled to the handle 3505. In this way movement of the handle 3505 relative to the shaft 3520 effects a corresponding movement of the actuator 3530.

This system, similarly to other systems, is configured to engage with an anchor cartridge 3510. The anchor cartridge in this configuration is configured, on receipt with the system, to be rotatable relative to the shaft 3520. A plurality of anchors may be disposed within the cartridge 3510, each being receivable within a seat 3520A, 3520B of the cartridge. On rotation of the cartridge relative to the shaft, individual ones of the seats are brought into alignment with the actuator 3530 and the shaft 3520. A pushing of the actuator via the handle causes the actuator to engage with a specific one of the retained anchors within the cartridge. Continued movement of the actuator towards the handle distal end 3522 of the shaft causes the engaged anchor to travel out of the cartridge, into the lumen of the shaft 3520 and then subsequently be displaced out of and away from the device. Once the actuator is retracted again, the cartridge is free to rotate and present another anchor for delivery.

To ensure alignment of a received cartridge relative to the other elements of the system, a platform 3515 on which the cartridge 3510 may be seated is provided. The platform defines a guide slot 3511 which is configured to engaged with an alignment member 3512 provided on the cartridge to ensure accurate receipt of the cartridge relative to the actuator and the shaft. The alignment member 3512 also acts as a locking member in a similar fashion to the locking member 2953 whereby the alignment member 3512 retains a spring 3513 which operably controls rotation of the received cartridge. This is shown in detail in FIG. 35B.

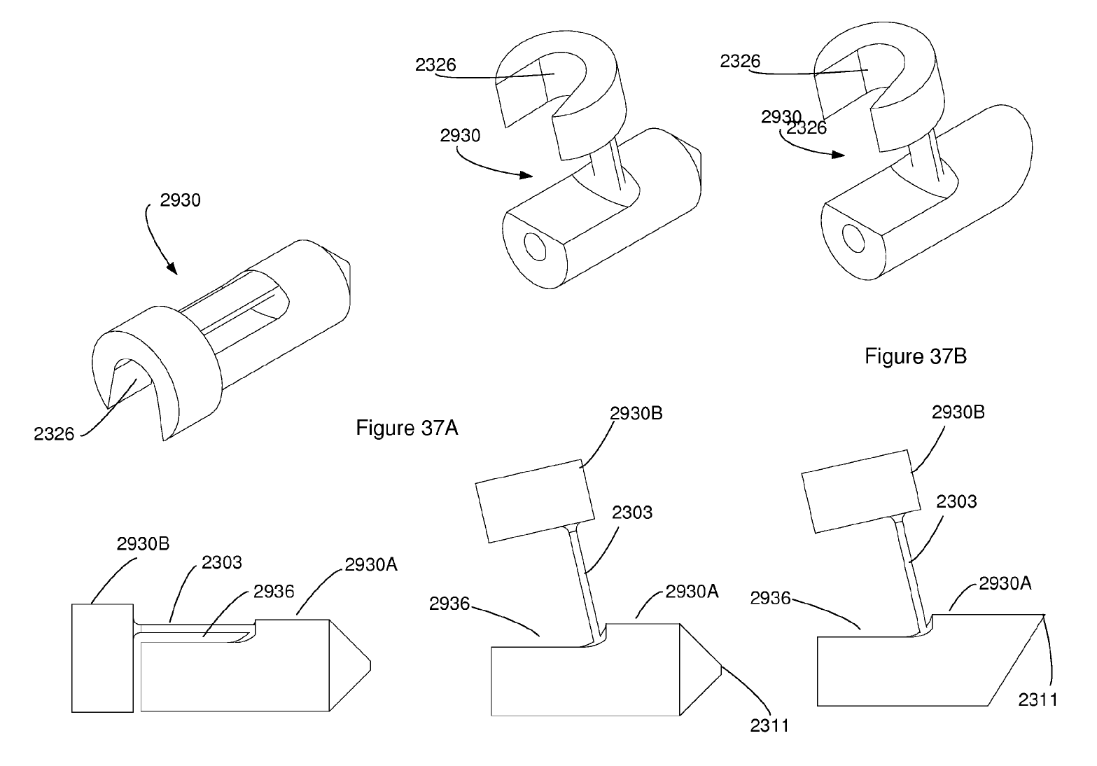

FIG. 37 illustrates a further embodiment of a two part anchor comprising a first part 2930A and 2930B connected by a interconnect member 2303. The main difference between this and previous embodiments is the detail of interconnect member 2303, which in this embodiment is shown having a rectangular profile. This may have advantages over a round profile in that where space is limited the strap can be configured to occupy less annular space than a circle of similar cross sectional area. The anchor 2930 is also shown with a cut-out portion 2936. This cut-out portion acts as a recess, into which the profile of the interconnect member sits during delivery to minimize the overall profile of the anchor.

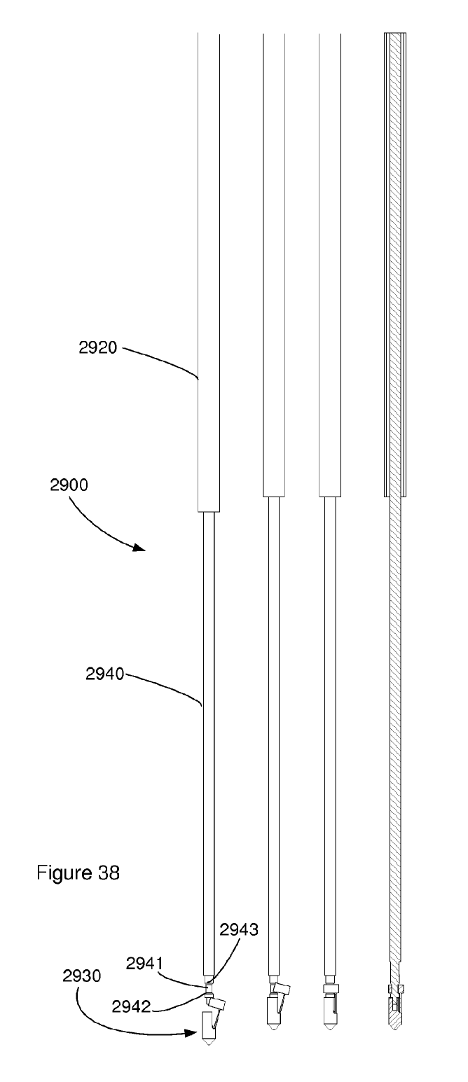

In FIG. 38, the anchor 2930 of FIG. 37 is shown with an actuator 2940, which feature a modification to previous embodiments. The actuator features a step down portion 2941. In providing this stepdown area a proximal step 2943 and distal step 2942 are created. When the trailing portion 2930B of the anchor interacts with the actuation 2940, the inner curved surface 2326 of the anchor is received onto the step down portion. In a scenario where the handle is accidently pulled proximal by the user as the anchor is being advanced through the shaft 2920, the distal step acts to prevent the anchor 2930 disengaging with the actuator 2940.

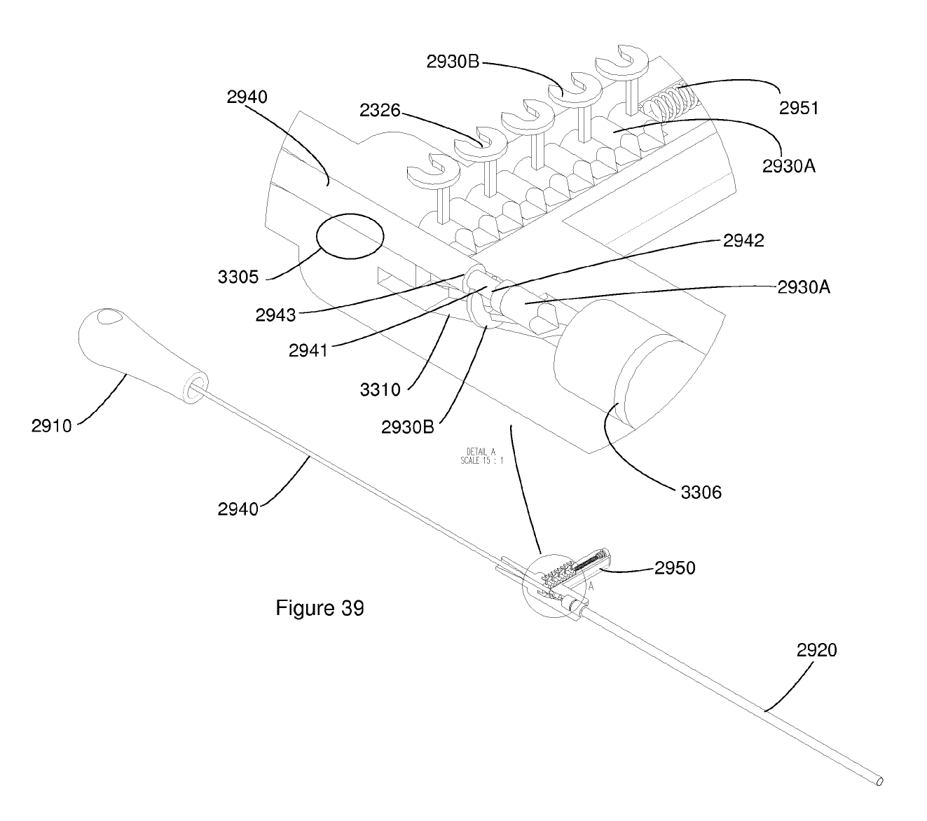

Furthermore a plurality of step down portions could be provided at varying distances from the distal tip of the actuator. Such a feature would be useful to allow the pusher interact and retain anchors where the length of the interconnect member is supplied in different lengths to suit certain applications as described earlier. An example of a device with this modification is shown in FIG. 39 where the actuator 2940 features a step down portion. The anchor second part 2930B is modified with a c-clip style second part, which sits around the actuator on delivery and engages with the step 2942 to prevent the anchor accidentally deploying if the pusher is retracted prior to exiting the shaft 2920. A single longer step could also be incorporated to facilitate compatibility with a variety of anchors of varying interconnect length.

A further modification could be made to the cartridge 2950 of FIG. 39 where a spring plunger, or other interference mechanism is provided to interact with the actuator pusher 2940 in the region proximal to the ramp 3310, identified in the drawing as 3305. This modification would provide a tactile stop when the actuator is retracted and the step down portion 2941 enters this zone, which alerts the user to stop retracting the pusher. The force of the spring plunger would be such that upon completion of the cartridge the pusher could be removed for use with additional cartridges if necessary. It will also be appreciated from a careful review of the ramp arrangement of FIG. 39 with that of FIG. 33 that a lead in portion to the ramp is larger in FIG. 39 to accommodate anchors with larger dimensioned second parts 2930B.

Similarly a spring plunger or other interference mechanism could be provided in the cartridge 2950 to interact with the annular recess 3306 of the shaft portion of the device. This would allow the user to quickly pull the shaft out of the cartridge and replace it with a shaft of modified length, for example, as described earlier. Alternately, the proximal end of the shaft 2922 could be ferromagnetic and be retained in the cartridge by magnets.

While not described in all embodiments, any one of the shafts may include the engagement portion previously described with reference to FIG. 19. As detailed above, this feature may be manufactured from a polymer and may be tacky or feature a textured surface to improve grip. Such a feature could be advantageously employed to prevent slippage of the device once a desire tack location is selected or to manipulate a hernia mesh into place and retain it in that position so long as the tip of the device remains in contact with the mesh.

Such an engagement portion may be provided separate to the shaft on which it is located. In this way after use, the engagement portion can be disposed of, the shaft re-sterilised and a new engagement portion located onto that sterilised shaft. The sleeve could be provided as a push fit, whereby the surface friction of it and the outer surface of shaft is superior to the types of forces it is likely to see in use. Alternately, the sleeve could be modified by the addition of protrusions on the silicone inner surface could be provided to cooperate with recesses which may be provided in the outer surface of the shaft to allow for fixation of the silicone sleeve. The following is an example of how the device of FIG. 29 may be used to fix a hernia mesh. First the user:
a. Attaches the cartridge to the shaft
b. Advances the actuator through the cartridge and shaft. The anchor is automatically loaded onto the actuator.
c. The user directs the shaft to the target location for anchor deployment and continues to advance until the tip of the anchor exits the shaft.
d. The actuator is advanced until the second part is suitably tensioned against the mesh or object being fixated
e. The actuator is then retracted until its tip is proximal to the next anchor at which point the next anchor is automatically or manually positioned for subsequent deployment.
f. Steps b to e are then repeated until the desired quality of anchors are deployed, if the cartridge is empty the user can load another and continue as before.

In this way it will be appreciated that while preferred arrangements have been described in an effort to assist in an understanding of the teaching of the present invention it will be appreciated that it is not intended to limit the present teaching to that described and modifications can be made without departing from the scope of the invention.

It will be appreciated that the exemplary arrangements or examples of devices have been described with reference to the Figures attached hereto. Where a feature or element is described with reference to one Figure, it will be understood that the feature or element could be used with or interchanged for features or elements described with reference to another Figure or example. The person of skill in the art, when reviewing the present teaching, will understand that it is not intended to limit the present teaching to the specifics of the illustrated exemplary arrangements as modifications can be made without departing from the scope of the present teaching.

The words comprises/comprising when used in this specification are to specify the presence of stated features, integers, steps or components but does not preclude the presence or addition of one or more other features, integers, steps, components or groups thereof.

1-72. (canceled)

73. An anchor delivery device comprising:
a handle coupled to a shaft, the shaft comprising a handle distal end of the shaft and a handle proximal end of the shaft, at least the handle distal end of the shaft being hollow and dimensioned to accommodate at least a portion of an anchor therein;
an actuator configured to effect a displacement of the anchor away from the shaft; and wherein
the anchor is a two part anchor, a first part being dimensioned to positively engage with the actuator during a delivery of the anchor out of and away from the shaft, and a second part configured to follow the first part during the delivery such that the first part is displaced away from the shaft prior to an exit of the second part away from the shaft, wherein the first part and the second part of the anchor are coupled to one another via a flexible member and further wherein the shaft is dimensioned to accommodate the first and second parts of the anchor, the shaft defining first and second guide channels for accommodating each of the first and second parts of the anchor and the device further comprising a biasing member for biasing the first and second parts into a common exit port from which they exit the shaft.

74. The device of claim 73 comprising a trigger coupled to the actuator, the trigger providing selective actuation of the actuator.

75. The device of claim 73 where the first part is coupled to the second part via an interconnect, the anchor configured such that the interconnect fits within the first part to minimise its deployment profile.

76. The device of claim 73 where the second part is in a plane substantially perpendicular to the first part.

77. The device of claim 73 where the second part has an at least partially circular portion in a plane substantially perpendicular to the first part.

78. The device of claim 73 where the second part is angled such that after deployment it is biased towards a target location.

79. The device of claim 73 wherein the actuator comprises a pushing rod, moveable within the shaft to effect the displacement of the anchor away from the shaft.

80. The device of claim 73 wherein the handle is hollow, an interior volume of the handle accommodating a geared mechanism providing for selective release of the anchor from the device.

81. The device of claim 74 wherein the trigger is housed within the handle, the trigger being moveable relative to the housing.

82. The device of claim 73 wherein the shaft is dimensioned to receive a plurality of anchors.

83. The device of claim 73 wherein the handle distal end of the shaft is moveable relative to the handle so as to expose the anchor.

84. The device of claim 73 wherein the handle distal end of the shaft is operably biased away from the handle.

85. The device of claim 73 comprising an engagement portion provided at a distal end of the shaft, the engagement portion being formed from a lower durometer material than the shaft.

86. The device of claim 85 wherein the engagement portion is removable from the shaft and provides a housing for one or more pre-loaded anchors.

87. The device of claim 73 comprising a resilient member configured to bias an anchor within the shaft so as to effect retention of the anchor.

88. The device of claim 73 wherein the actuator and first part of the anchor are configured to engage with one another during a delivery of the anchor out of the shaft.

89. The device of claim 88 wherein the actuator is receivable within at least a portion of the first part of the anchor or wherein the anchor is receivable within at least a portion of the actuator.

90. The device of claim 73 configured to receive a cartridge, the cartridge comprising a plurality of anchors, and the actuator is configured to selectively displace individual anchors from a received cartridge, such that a first anchor is delivered from the shaft prior to engagement of a second anchor with the actuator.

91. The device of claim 90 wherein each of the shaft and the handle are mateable with a received cartridge, the cartridge operably being provided between each of the handle and the shaft.

92. The device of claim 73 wherein actuator is coupled to the handle and moveable within the shaft in response to a movement of the handle relative to the shaft.

93. A anchor delivery device comprising:
a hollow shaft defining a first channel and a second channels, at least the first channel dimensioned for accommodating at least a portion of a surgical anchor;
a biasing member for directing the at least a portion of the surgical anchor from the first channel into the second channel; and
an actuator for effecting displacement of the anchor from the second channel to exit the delivery device.

94. The device of claim 93, the shaft comprising a handle distal end and a handle proximal end, the handle distal end being moveable relative to the handle to expose the anchor.

95. The device of claim 93 wherein the actuator is at least partially displaced in the second channel.

96. The device claim 93 wherein the actuator extends through the second channel.

97. The device of claim 93 wherein each of the first and second channels are dimensioned for accommodating at least a portion of the same surgical anchor.

98. A anchor delivery device comprising:
a shaft having a proximal end and a distal end;
an engagement portion provided at the distal end of the shaft and defining a channel for operably receiving at least a portion of a surgical anchor, the engagement portion being formed from a lower durometer material than the shaft; and
an actuator for effecting displacement of the anchor from the channel to exit the delivery device.

99. The device of claim 98 wherein the channel is dimensioned to receive a tubular anchor.

100. The device of claim 98 wherein the engagement portion is dimensioned to simultaneously receive a plurality of anchors.

101. The device of claim 98 wherein the engagement portion is removable from the shaft.

102. A multi-part surgical anchor delivery kit, the kit comprising a handle coupled to an actuator, a shaft co-operable with the actuator and through which the actuator may pass and a separate cartridge comprising a plurality of anchors, the handle, actuator and shaft being configured to receive and engage with the cartridge to define a surgical anchor delivery tool, and wherein the shaft comprises a handle proximal end and a handle distal end, the handle proximal end being configured to operably receive an anchor displaced from the cartridge, the actuator being configured to couple with and drive the anchor through the shaft so as to be displaced from the kit through a port provided at the handle distal end of the shaft.

103. The kit of claim 102 wherein the shaft distal end comprises a pair of wings, the wings being tapered outwardly from the shaft distal end to the shaft proximal end, the cartridge defines a first and second recess dimensioned to accommodate the wings, such that when the cartridge coupled with the handle, the wings slide inside the cartridge to effect securement of the cartridge to the handle.

104. The kit of claim 102 wherein the cartridge comprises a pair of protrusions configured to couple with corresponding slots provided on the handle.

105. The kit of claim 102 wherein the cartridge comprises a biasing member configured to bias individual anchors into alignment with the actuator.

106. The kit of claim 102 wherein the cartridge is rotatable relative to the shaft.

107. The kit of claim 102 wherein the anchors are two part anchors, a first part having a different geometrical form to a second part.

108. The kit of claim 102 wherein the first part and second part are coupled by an interconnect, having a different geometrical form to each of the first part and the second part.

109. The kit of claim 108 configured to receive anchors of different types, a distance between each of the first part and second part as defined by the interconnect of each anchor determining fixation properties of the anchor.

110. The kit of claim 109 wherein the interconnect is configured to maintain a tensile strength of at least 20N.

111. Then kit of claim 109 where the interconnect is configured in a rectangular profile to maximise its tensile strength while minimising its delivery profile.

112. The kit of claim 111 where the interconnect has a rectangular profile with a length of 0.75 to 1.5 mm and a width of 0.25 to 0.75 mm.

113. The kit of claim 102 wherein the cartridge is provided separate to the handle and shaft.

114. A method of deploying a surgical anchor within an abdominal cavity, the method comprising:
a. providing a device as claimed in claim 73;
b. advancing the actuator into engagement with an anchor so as to load the anchor onto the actuator;
c. directing the shaft to a target location for anchor deployment, a mesh being provided at the target location;
d. advancing the actuator through the shaft until a tip of the anchor exits the shaft;
e. advancing the actuator until a second part of the anchor is tensioned against the mesh; and
f. retracting the actuator to a location where it can engage with a second anchor.

115. The method of claim 114 wherein the device comprises a plurality of tacks provided in a cartridge, the cartridge being removable from the device.

116. The method of claim 115 comprising replacing a cartridge during a surgical procedure without having to remove the shaft from the abdominal cavity.
